(19) United States
(12) Patent Application Publication
(10) Pub. No.: US 2003/0194391 A1
(43) Pub. Date: Oct. 16, 2003

(54) Vaccine

(76) Inventors: Claire Ashman, Stevenage (GB); James Scott Crowe, Stevenage (GB); Jonathan Henry Ellis, Stevenage (GB); Alan Peter Lewis, Stevenage (GB)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/233,902

(22) Filed: Sep. 3, 2002

Related U.S. Application Data
(63) Continuation-in-part of application No. PCT/GB02/00900, filed on Mar. 1, 2002.

(30) Foreign Application Priority Data
Mar. 3, 2001 (GB) 0105360.2

(51) Int. Cl. A61K 48/00; A61K 38/20; C07H 21/04; C12P 21/02; C12N 5/06; C07K 14/54

(52) U.S. Cl. 424/85.2; 435/69.5; 435/320.1; 435/325; 530/351; 536/23.5; 514/44

(57) ABSTRACT

The present invention relates to an isolated polypeptide useful for immunisation against self-antigens. In particular the invention relates to a self-protein that is capable of raising auto-antibodies when administered in vivo. The invention particularly relates to rendering human cytokines immunogenic in humans. The invention further relates to pharmaceutical compositions comprising such compounds and their use in medicine and to methods for their production.

The present invention relates to an isolated polypeptide useful for immunisation against self-antigens. In particular the invention relates to a self-protein that is capable of raising auto-antibodies when administered in vivo. The invention particularly relates to rendering human cytokines immunogenic in humans. The invention further relates to pharmaceutical compositions comprising such compounds and their use in medicine and to methods for their production.

### BACKGROUND OF THE INVENTION

Asthma is a chronic lung disease, caused by inflammation of the lower airways and is characterised by recurrent breathing problems. Airways of patients are sensitive and swollen or inflamed to some degree all the time, even when there are no symptoms. Inflammation results in narrowing of the airways and reduces the flow of air in and out of the lungs, making breathing difficult and leading to wheezing, chest tightness and coughing. Asthma is triggered by super-sensitivity towards allergens (e.g. dust mites, pollens, moulds), irritants (e.g. smoke, fumes, strong odours), respiratory infections, exercise and dry weather. The triggers irritate the airways and the lining of the airways swell to become even more inflamed, mucus then clogs up the airways and the muscles around the airways tighten up until breathing becomes difficult and stressful and asthma symptoms appear.

COPD is an umbrella term to describe diseases of the respiratory tract, which shows similar symptoms to asthma and is treated with the same drugs. COPD is characterised by a chronic, progressive and largely irreversible airflow obstruction. The contribution of the individual to the course of the disease is unknown, but smoking cigarettes is thought to cause 90% of the cases. Symptoms include coughing, chronic bronchitis, breathlessness and respiratory injections. Ultimately the disease will lead to severe disability and death.

As a result of the various problems associated with the production, administration and tolerance of monoclonal antibodies there is an increased focus on methods of instructing the patient's own immune system to generate endogenous antibodies of the appropriate specificity by means of vaccination. However, mammals do not generally have high-titre antibodies against self-proteins present in serum, as the immune system contains homeostatic mechanisms to prevent their formation. The importance of these tolerance mechanisms is illustrated by diseases like myasthenia gravis, in which auto-antibodies directed to the nicotinic acetylcholine receptor of skeletal muscle cause weakness and fatigue (Drachman, 1994, N Engl J Med 330:1797-1810). There is therefore a need for a vaccine approach which is able to circumvent antibody tolerance mechanisms without inducing auto-antibody-mediated pathology.

A number of techniques have been designed with the aim of breaking B cell tolerance without necessarily inducing unacceptable autoimmune toxicity. However, all have significant drawbacks.

One technique involves chemically cross-linking either the self-protein (or peptides derived from it) to a highly immunogenic carrier protein, such as keyhole limpet haemocyanin (Antibodies: A laboratory manual” Harlow, E and Lane D. 1988. Cold Spring Harbor Press). This approach is a variant of the widely used hapten-carrier system for raising antibodies to poorly immunogenic targets, such as low-molecular weight chemical compounds. However, the process of chemical conjugation can destroy potentially valuable epitopes, and much of the evoked antibody response is directed at the carrier protein. Furthermore this approach is only applicable to protein vaccination, and is not compatible with nucleic acid immunogens.

A variant on the carrier protein technique involves the construction of a gene encoding a fusion protein comprising both carrier protein (for example hepatitis B core protein) and self-protein (The core antigen of hepatitis B virus as a carrier for immunogenic peptides”, Biological Chemistry. 380(3):277-83, 1999). The fusion gene may be administered directly as part of a nucleic acid vaccine. Alternatively, it may be expressed in a suitable host cell in vitro, the gene product purified and then delivered as a conventional vaccine, with or without an adjuvant. However, fusing a large carrier protein to the self-protein can constrain or distort the self-protein's conformation, reducing its efficiency in evoking antibodies cross-reactive with the native molecule. Also, like the traditional cross-linked carrier systems, much of the antibody response is directed to the carrier part of the fusion. Anti-carrier responses may limit the effectiveness of subsequent booster administrations of vaccine or increase the chance of allergic or anaphylactic reactions.

A more refined approach has been described by Dalum and colleagues wherein a single class II MHC-restricted epitope is inserted into the target molecule. They demonstrated the use of this method to induce antibodies to ubiquitin (Dalum et al, 1996, J Immunol 157:4796-4804; Dalum et al, 1997, Mol Immunol 34:1113-1120) and the cytokine TNF (Dalum et al, 1999, Nature Biotech 17:666-669). As a result, all T cell help must arise either from this single epitope or from junctional sequences. While this approach may work well in subjects possessing the appropriate MHC class II haplotype for which the vaccine was designed, or indeed those fortunate enough to have class II molecules capable of binding junctional epitopes, in any normal outbred population, such as those typical of humans, there will be a significant portion of the population for whom the vaccine will not work. Additionally, since the inserted epitope is typically from a quite unrelated protein, such as ovalbumin or lysozyme, it is likely that the additional sequence will to some degree interfere with the folding of the target protein, preventing the adoption of a fully native conformation of the target protein.

In contrast to all of the above, the present invention provides a multiplicity of potential T cell epitopes, yet retains the target molecule in a conformation close to the native form. These properties allow the vaccines of the present invention to be effective immunogens in complex outbred populations, such as those composed of human patients. These properties are achieved by rendering a mutation in a self-protein to produce a sequence at that point which can be found in an analogous protein.

A number of recent papers have defined a critical role for the Th2 cytokine IL-13 in driving pathology in the ovalbumin model of allergenic asthma (Wills-Karp et al, 1998; Grunig et al, 1998). In this work, mice previously sensitised to ovalbumin were injected with a soluble IL-13 receptor which binds and neutralises IL-13. Airway hyper-responsiveness to acetylcholine challenge was completely ablated in the treated group. Histological analysis revealed that treated mice had reversed the goblet-cell metaplasia seen in controls. In complementary experiments, lung IL-13 levels were raised by over-expression in a transgenic mouse or by installation of protein into the trachea in wild-type mice. In both settings, airway hyper-responsiveness, eosinophil invasion and increased mucus production were seen (Zhu et al, 1999). These data show that IL-13 activity is both necessary and sufficient to produce several of the major clinical and pathological features of allergic asthma in a well-validated model.

A vaccine capable of directing a neutralising response to IL-13 would therefore constitute a useful therapeutic for the treatment of allergic asthma in humans. It would also have application in the treatment of certain helminth infection-related disorders (Brombacher, 2000) and diseases where IL-13 production is implicated in fibrosis (Chiaramonte et al, 1999), such as chronic obstructive pulmonary disease. The present invention addresses this need.

The concepts and principles of the invention are thus set forth with respect to IL-13, but can be applied to any mammalian self-protein having an analogous protein in a second species.

### SUMMARY OF THE INVENTION

The present invention provides an isolated polypeptide which is at least 30% but less than 100% identical to a human protein which polypeptide (a) contains at least one mutation which is characteristic of an analogous non human protein;

and b) is capable of raising antibodies in humans and

(c) is sufficiently structurally similar to the human protein that the antibodies bind to both the human protein and the polypeptide; and (d) wherein the polypeptide is not an antibody.

Thus the invention provides in one embodiment; a protein having B cell epitopes from a mammalian self-antigen and a mutation that gives rise to a sequence of an analogous protein from a second mammalian species, such that the protein is able to raise in the species from which the B-cell epitopes are derived, an immune response that recognises the native protein from which the B-cell epitopes are derived.

Preferably the sequence of the analogous protein is more than 5, more preferably greater than 8 contiguous amino acids. Thus the protein of the present invention contains a sequence that is identical to the analogous sequence for at least 5, preferably at least 8 consecutive amino acids. In an alternative embodiment a protein is provided having B cell epitopes of a self protein which are grafted by substitution, into a framework of an analogous protein from a second mammalian species such that the protein is able to raise in the species in which the B cell epitopes are derived an immune response that recognises the natural protein from which the B-cell epitopes are derived.

It will be appreciated that the protein of the present invention are not an antibody.

The immune response raised is preferably an antibody response, most preferably a neutralising antibody response.

In general the mutation is introduced preferably into the non-surface exposed region of the molecule, such that surface exposed regions are conserved. Surface exposed regions are accessible to the immune-system and consequently often contain B-cell epitopes. Accordingly the present invention provides a protein comprising conserved surface exposed regions of a self protein, and a mutation introduced into the non-surface exposed region, said mutation giving rise to a sequence of an analogous protein such that the protein is able to raise an immune response to the self-protein arises in the species from which the self-protein is derived.

The self protein is preferably a human protein, but can be a protein from any mammal in which it is desired to raise an auto immune response to. The immune response is preferably specific to the native protein and immunogen of the invention. That is having minimal cross-reactivity or neutralising capacity with respect to other self proteins.

The self antigen is preferably a cytokine, more preferably a 4 helical cytokine, more preferably IL-4 or IL-13, most preferably IL-13. Thus in a preferred embodiment of the present invention there is provided a chimaeric protein comprising B cell epitopes from Human IL-13 presented in a murine IL-13 back bone. Such a construct is capable of raising a specific anti IL-13 antibody response in humans. Such a construct is shown in FIG. 9 (seq: ID No 21 and 22). Similarly an IL-4 construct comprising human IL-surface regions and murine framework is presented in FIG. 13 (Seq ID: No 25).

The invention also provides:

an expression vector which comprises a polynucleotide of the invention and which is capable of expressing a polypeptide of the invention;

a host cell comprising an expression vector of the invention; a method of producing a polypeptide of the invention which method comprises maintaining a host cell of the invention under conditions suitable for obtaining expression of the polypeptide and isolating the said polypeptide:

a vaccine composition comprising a polypeptide or polynucleotide of the invention and a pharmaceutically acceptable carrier.

In another aspect, the invention provides a method for the design and preparation of a polypeptide according to the invention which method comprises:

1. identification of one or more regions of a self, typically human, protein against which an antibody response is desired.

2. identification of the amino-acid sequence of the self protein.

3. identification of the amino-acid sequence of an analogous protein construction by recombinant DNA techniques of a chimaeric molecule containing at least one target region identified in step 1, whose amino-acid sequence is taken from the sequence identified in step 2, and

sufficient amino-acids from the sequence(s) identified in step 3 to enable the resulting protein to fold into a shape similar to that the self protein such that the mutated protein can raise an immune response that recognises the self protein.

In a further aspect of the present invention there is provided a method of treatment of asthma comprising the administration to a patient in need thereof, or a vaccine which is capable of generating in said host and anti-IL-13 immune response. Also provided by the present invention are vaccines for use in the method of treatment of asthma.

### DESCRIPTION OF FIGURES

GST=glutathione S-transferase, rmIL-13=recombinant mouse IL-13, rhIL-13=recombinant human IL-13, cIL-13=chimaeric IL-13

FIG. 1. Sequence of mouse chimaeric IL-13 vaccine construct. Underlined aminoacid symbols denote sequence human IL-13, unmodified symbols are from murine IL-13.

FIG. 2. Analysis of GST cIL-13 by 4-20% Tris-glycine SDS-PAGE gel (Novex), stained for total protein with Coomassie Blue.

FIG. 3. Western blot analysis of GST-cIL-13.

FIG. 4. ELISA analysis of cIL-13 and GST-cIL-13 interaction with anti-mIL-13 polyclonal antibody, anti-hIL-13 polyclonal antibody and anti-GST polyclonal antibody.

FIG. 5. ELISA analysis of the interaction of cIL-13 and GST-cIL-13 with the mIL-13 receptors, mIL-13Rα1 and mIL-13Rα2.

FIG. 6. Anti-phospho-STAT6 Western blot of A549 lysates.

FIG. 7. Antibody responses induced by immunisation with GST-cIL-13 (mouse F5) or cIL-13 (mouse E5).

FIG. 8. Anti-phospho-STAT6 Western blot analysis of A549 lysates.

FIG. 9 Chimaeric IL-13 vaccine for use in humans. Underlined aminoacid symbols denote sequence found in murine IL-13, unmodified symbols are from human IL-13.

FIG. 10. Anti-mouse IL-13 antibody profiles follow administration of cIL-13 in combination with various adjuvants.

FIG. 11. Serum neutralisation capacity of mice following administration of cIL-13.

FIG. 12. Alternative cIL-13 for use as a mouse immunogen.

FIG. 13. Chimaeric IL-4 for use in human anti IL-4 vaccine.

### DETAILED DESCRIPTION OF THE INVENTION

Throughout this specification and the appended claims, unless the context requires otherwise, the words “comprise” and “include” or variations such as “comprising”, “comprises”, “including”, “includes” etc., are to be construed inclusively, that is, use of these words will imply the possible inclusion of integers or elements not specifically recited.

As described herein, the present invention relates isolated polypeptides and isolated polynucleotides. In the context of this invention the term “isolated” is intended to convey that the polypeptide or polynucleotide is not in its native state, insofar as it has been purified at least to some extent or has been synthetically produced, for example by recombinant methods, or mechanical synthesis. The term “isolated” therefore includes the possibility of the polypeptides or polynucleotides being in combination with other biological or non-biological material, such as cells, suspensions of cells or cell fragments, proteins, peptides, expression vectors, organic or inorganic solvents, or other materials where appropriate, but excludes the situation where the polynucleotide is in a state as found in nature.

An advantage of the invention is that the polypeptide of the invention contains regions of the self, eg human protein against which an antibody response is desired, in association with regions characteristic of an analogous protein which are sufficiently different to the human protein to provide excellent T cell help, but yet are optimised by evolution to fold into a shape highly similar to the human protein. This allows antibodies to be raised that recognise the self antigen. Typically, the immune response raised includes the raising of a neutralising antibody response.

The human protein according to the invention may be a full length protein encoded by the human genome or a domain or sub-unit of a full length protein encoded by the human genome. Where it is desired to raise neutralising antibodies against a functional domain of the self antigen—or a receptor binding domain a chimaeric antigen involving only these regions may be prepared. Thus the exposed region of such a domain, or the B cell epitopes of such a domain are conserved and mutation of an analogues protein is introduced in the non-B cell epitope or surface exposed domains.

The term ‘protein’ is intended to include, for example, shorter sequences of amino acid residues which may be referred to as peptides, such as neuropeptides. The human protein will typically be the subject of post-translational modification such as glycosylation, proteolytic cleavage, phosphorylation, and others well known to those skilled in the field. The human protein is preferably a cytokine, a hormone, a growth factor or an extracellular protein, more preferably a 4-helical cytokine, most preferably IL-13. Cytokines include, for example, IL1, IL2, IL3, IL-4, IL5, IL6, IL7, IL8, IL9, IL10, IL11, IL12, IL13, IL14, IL15, IL16, IL17, IL18, IL20, IL21, IL25, TNF, TGF, GMCSF, MCSF and OSM. 4-helical cytokines include IL2, IL3, IL-4, IL5, IL13, GMCSF and MCSF. Hormones include, for example, luteinising hormone (LH), follicle stimulating hormone (FSH), chorionic gonadotropin (CG), VGF, GHrelin, agouti, agouti related protein and neuropeptide Y. Growth factors include, for example, VEGF. Extracellular proteins include, for example, APP or B-amyloid.

An analogous protein is one which is orthologous or paralogous to the self-protein, eg human protein, wherein an orthologous protein can be traced by descent to a common ancestor of the different organisms and is therefore likely to perform similar conserved functions in the different organisms. Thus an orthologous gene means genes which are so similar in sequence they have originated from a single ancestral gene and thus are an equivalent gene in a different species and have evolved from a common ancestor by specification. In particular in humans the orthologous protein is a structually equivalent molecule in a non human mammal. A paralogous protein is one which appears in more than one copy in a given organism by a duplication event (Venter, Science; 1336, vol 291; 2001), that is homologous sequence (sharing a common evolutionary ancestors) that have diversed by gene duplication. Preferably the analogous protein is an orthologue. An orthologous protein will typically have the same name as the human protein and will typically perform the same function, for example murine IL-13 is the orthologue to human IL-13. The analogous protein is typically mammalian or avian, for example, bovine, ovine, rodent, such as murine, porcine, simian, feline, canine or human. Preferably the analogous protein is murine. Thus in the context of the present invention, Murine IL-13 is an analogous (and orthologous) protein to human IL-13. Similiarly simian IL-4 is an analogous (and orthologous) protein to human IL-4.

The polypeptide of the invention preferably comprises 2, 3, 4, 5, 6, 7, 8, 9, 10, 11 or more mutations characteristic of an analogous protein. More preferably the polypeptide comprises at least three mutations. Each mutation may be characteristic of the same or different analogous proteins. Thus a first mutation might be characteristic of a murine analogue and a second mutation might be characteristic of a simian analogue. According to one feature, the polypeptide comprises at least three mutations, where each mutation is characteristic of a different analogue. Preferably, however, each mutation is characteristic of the same analogue. A mutation is a change in the amino acid sequence of the protein and includes, for example, deletions, insertions and substitutions. Preferably the mutation is a substitution. Preferably more than one amino acids are replaced in each non-surfaced exposed region.

A mutation which is characteristic of an analogous protein is one which results in the sequence of the human protein being closer in identity to the sequence of the analogous protein after the mutation has been made to the human protein. For example when the human sequence is ProProArgVal and the murine analogue has the sequence ProProTyrVal, a mutation characteristic of the analogous protein is to substitute Arg for Tyr. Preferably the mutation is not made in residues which are surface residues in native folded active protein in aqueous solution under physiological conditions. These surface residues particular those forming loop structures are often B cell epitopes and it is preferred that all of these regions are conserved. The mutations thus introduced have the function of breaking the tolerance of the self-protein and being immunogenic in the species that the non-mutated protein is derived from.

In an embodiment the polypeptides of the invention are at least 30% and less than 100% identical to a human protein, preferably over the whole length of the human protein. Preferably the polypeptides are at least 40%, for example at least 50% identical to the human protein. More preferably the polypeptides are at least 60%, for example, at least 70% identical to the human protein. Most preferably the polypeptides are at least 85% identical to the human protein, for example, about 90% identical. Such proteins are capable of raising an immune response in humans that recognise the human protein.

For example, the UWGCG Package provides the BESTFIT program which can be used to calculate homology (for example used on its default settings) (Devereux et al (1984) Nucleic Acids Research 12, p387-395). The PILEUP and BLAST algorithms can be used to calculate homology or line up sequences (typically on their default settings), for example as described in Altschul (1993) J. Mol. Evol. 36:290-300; Altschul et al (1990) J. Mol. Biol. 215:403-10.

Software for performing BLAST analyses is publicly available through the National Centre for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, 1990). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands, when the program is being used on polynucleotides.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

The successful design of a polypeptide according to the present invention can be verified for example by demonstrating that, when expressed in an appropriate host cell, the polypeptide adopts a conformation sufficiently similar to that of the self protein that antibodies are generated which are cross-reactive with the native self protein. This may be shown using immunological techniques, such as binding of monoclonal or polyclonal antibodies in ELISA, or by physicochemical techniques such as circular dichroism, or by crystallographic techniques such as X-ray crystallography or by computer modelling, or by numerous other approaches well known to those skilled in the art.

Further confirmation of a successful design can be obtained by administering the resulting polypeptide in a self-context in an appropriate vaccination regime, and observing that antibodies capable of binding the protein are induced. This binding may be assessed through use of ELISA techniques employing recombinant or purified native protein, or through bioassays examining the effect of the protein on a sensitive cell or tissue. A particularly favoured assessment is to observe a phenomenon causally related to activity of the protein in the intact host, and to determine whether the presence of antibodies induced by the methods of the invention modulate that phenomenon. Thus a protein of the present invention will be able to raise antibodies to the native antigen in the species from which the native protein is derived.

The polypeptide of the invention may be further modified by mutation, for example substitution, insertion or deletion of amino-acids in order to add desirable properties (such as the addition of a sequence tag that facilitates purification or increase immunogenicity) or remove undesirable properties (such as an unwanted agonistic activity at a receptor) or trans-membrane domains. In particular the present invention specifically contemplates fusion partners that ease purification such as poly histidine tags or GST expression partners that enhance expression.

In a preferred embodiment there is provided a human IL-13 having one or more of the following mutations or a conservative substitution thereof characteristic of mouse IL-13. The following numbering refers to IL-13 expressed with its signal sequence in E.coli.

| | | | | | |
|---|---|---|---|---|---|
| R | → | K | at | position | 30 |
| V | → | S | at | position | 37 |
| Y | → | F | at | position | 63 |
| A | → | V | at | position | 65 |
| E | → | D | at | position | 68 |
| E | → | Y | at | position | 80 |
| K | → | R | at | position | 81 |
| M | → | I | at | position | 85 |
| G | → | H | at | position | 87 |
| Q | → | H | at | position | 113 |
| V | → | I | at | position | 115 |
| D | → | K | at | position | 117 |

More preferably the human IL-13 comprises at least two preferably at least 3,4,5,6 or more of the above mutations or a conservative substitution thereof. It is preferred that all twelve mutations are present.

A “conservative substitution” is one in which an amino acid is substituted for another amino acid that has similar properties, such that one skilled in the art of peptide chemistry would expect the secondary structure and hydropathic nature of the polypeptide to be substantially unchanged.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated that various changes may be made in the peptide sequences of the disclosed compositions, or corresponding DNA sequences which encode said peptides without appreciable loss of their biological utility or activity.

In making such changes, the hydropathic index of amino acids may be considered. The importance of the hydropathic amino acid index in conferring interactive biologic function on a protein is generally understood in the art (Kyte and Doolittle, 1982, incorporated herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of its hydrophobicity and charge characteristics (Kyte and Doolittle, 1982). These values are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity. U.S. Pat. No. 4,554,101 (specifically incorporated herein by reference in its entirety), states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine. These are preferred conservative substitutions.

Amino acid substitutions may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his.

In a preferred embodiment, the mutated IL-13 of the present invention comprises one or more of the following sequences or a variant thereof comprising a conservative substitution:

| | |
|---|---|
| L K E L I E E L S N; | (SEQ ID No 1) |
| F C V A L D S L; | (SEQ ID No 2) |
| A I Y R T Q R I L H G; | (SEQ ID No 3) |
| K I E V A H F I T K L L; | (SEQ ID No 4) |

Human IL-13 vaccines of the present invention comprise chimaeric molecules that have the sequence of an orthologous IL-13, the B-cell epitopes of which have been substituted with the human equivalent regions. Accordingly, the present invention provides immunogens for use in vaccines against asthma comprising a non-human IL-13 amino acid sequence, which further comprises within said sequence at least one of the following human sequences:

GPVPPSTA
ITQNQKAPLCNGSMVWSINLTAGM
INVSGCS
FCPHKVSAGQFSSLHVRDT
LHLKKLFREGRFN

The polypeptide of the invention is encoded by polynucleotides of the invention. A person skilled in the art will readily be able to determine the sequence of the polynucleotide which encodes the polypeptide by applying the genetic code. Once the required nucleic acid sequence has been determined, the polynucleotide with the desired sequence can be produced as described in the examples. A skilled person will readily be able to adapt any parameters necessary, such as primers and PCR conditions. It will also be understood by a person skilled in the art that, due to the degeneracy of the genetic code, there is potentially more than one polynucleotide which encodes a polypeptide of the invention.

The polynucleotide of the invention is typically RNA, for example mRNA, or DNA, for example genomic DNA, cDNA or synthetic DNA. Preferably the polynucleotide is DNA. Particularly preferably it is cDNA.

The present invention further provides an expression vector, which is a nucleic acid construct, comprising the polynucleotide of the invention. Additionally, the nucleic acid construct will comprise appropriate initiators, promoters, enhancers and other elements, such as for example, polyadenylation signals, which may be necessary, and which are positioned in the correct orientation, in order to allow for protein expression within a mammalian cell.

The promoter may be a eukaryotic promoter for example a CD68 promoter, Gal1, Gal10, or NMT1 promoter, a prokaryotic promoter for example Tac, Trc, or Lac, or a viral promoter, for example the cytomegalovirus promoter, the SV40 promoter, the polyhedrin promoter, the P10 promoter, or the respiratory syncytial virus LTR promoter. Preferably the promoter is a viral promoter. Particularly preferred is when the promoter is the cytomegalovirus immediate early promoter, optionally comprising exon 1 from the HCMV IE gene.

The transcriptional regulatory elements may comprise enhancers, for example the hepatitis B surface antigen 3′untranslated region, the CMV enhancer; introns, for example the CD68 intron, or the CMV intron A, or regulatory regions, for example the CMV 5′ untranslated region.

The polynucleotide is preferably operably linked to the promoter on the nucleic acid construct such that when the construct is inserted into a mammalian cell, the polynucleotide is expressed to produce a encoded polypeptide.

The nucleic acid construct backbone may be RNA or DNA, for example plasmid DNA, viral DNA, bacterial DNA, bacterial artificial chromosome DNA, yeast artificial chromosome DNA, synthetic DNA It is also possible for the nucleic acid construct to be artificial nucleic acid, for example phosphorothioate RNA or DNA. Preferably the construct is DNA. Particularly preferred is when it is plasmid DNA.

The present invention further provides a host cell comprising an expression vector of the invention. Such cells include transient, or preferably stable higher eukaryotic cell lines, such as mammalian cells or insect cells, using for example a baculovirus expression system, lower eukaryotic cells, such as yeast or prokaryotic cells such as bacterial cells. Particular examples of cells which may be modified by insertion of vectors encoding for a polypeptide according to the invention include mammalian HEK293T, CHO, HeLa, NS0 and COS cells. Preferably the cell line selected will be one which is not only stable, but also allows for mature glycosylation of a polypeptide. Expression may be achieved in transformed oocytes. A polypeptide of the invention may be expressed in cells of a transgenic non-human animal, preferably a mouse or expressed into the milk of larger mammals, such as goats, sheep and cows. A transgenic non-human animal expressing a polypeptide of the invention is included within the scope of the invention. A polypeptide of the invention may also be expressed in Xenopus laevis oocytes.

The present invention also includes pharmaceutical or vaccine compositions, which comprise a therapeutically effective amount of nucleic acid construct or polypeptide of the invention, optionally in combination with a pharmaceutically acceptable carrier, preferably in combination with a pharmaceutically acceptable excipient such as phosphate buffered saline (PBS), saline, dextrose, water, glycerol, ethanol, liposomes or combinations thereof. The vaccine composition may alternatively comprise a therapeutically effective amount of a nucleic acid construct of the invention, formulated onto metal beads, preferably gold beads. The vaccine composition of the invention may also comprise an adjuvant, such as, for example, in an embodiment, imiquimod, tucaresol or alum.

Protein adjuvant formulations are preferred as these induce high titre antibody responses.

Preferably the adjuvant is administered at the same time as of the invention and in preferred embodiments are formulated together. Such adjuvant agents contemplated by the invention include, but this list is by no means exhaustive and does not preclude other agents: synthetic imidazoquinolines such as imiquimod [S-26308, R-837], (Harrison, et al. ‘Reduction of recurrent HSV disease using imiquimod alone or combined with a glycoprotein vaccine’, Vaccine 19: 1820-1826, (2001)); and resiquimod [S-28463, R-848] (Vasilakos, et al. ‘Adjuvant activites of immune response modifier R-848: Comparison with CpG ODN’, Cellular immunology 204: 64-74 (2000).), Schiff bases of carbonyls and amines that are constitutively expressed on antigen presenting cell and T-cell surfaces, such as tucaresol (Rhodes, J. et al. ‘Therapeutic potentiation of the immune system by costimulatory Schiff-base-forming drugs’, Nature 377: 71-75 (1995)), cytokine, chemokine and co-stimulatory molecules, Th1 inducers such as interferon gamma, IL-2, IL-12, IL-15 and IL-18, Th2 inducers such as IL-4, IL-5, IL-6, IL-10 and IL-13 other chemokine and co-stimulatory genes such as MCP-1, MIP-1 alpha, MIP-1 beta, RANTES, TCA-3, CD80, CD86 and CD40L, other immunostimulatory targeting ligands such as CTLA-4 and L-selectin, apoptosis stimulating proteins and peptides such as Fas, (49), synthetic lipid based adjuvants, such as vaxfectin, (Reyes et al., ‘Vaxfectin enhances antigen specific antibody titres and maintains Th1 type immune responses to plasmid DNA immunization’, Vaccine 19: 3778-3786) squalene, alpha-tocopherol, polysorbate 80, DOPC and cholesterol, endotoxin, [LPS], Beutler, B., ‘Endotoxin, ‘Toll-like receptor 4, and the afferent limb of innate immunity’, Current Opinion in Microbiology 3: 23-30 (2000)); CpG oligo- and di-nucleotides, Sato, Y. et al., ‘Immunostimulatory DNA sequences necessary for effective intradermal gene immunization’, Science 273 (5273): 352-354 (1996). Hemmi, H. et al., ‘A Toll-like receptor recognizes bacterial DNA’, Nature 408: 740-745, (2000) and other potential ligands that trigger Toll receptors to produce Th1-inducing cytokines, such as synthetic Mycobacterial lipoproteins, Mycobacterial protein p19, peptidoglycan, teichoic acid and lipid A.

Certain preferred adjuvants for eliciting a predominantly Th1-type response include, for example, a Lipid A derivative such as monophosphoryl lipid A, or preferably 3-de-O-acylated monophosphoryl lipid A. MPL® adjuvants are available from Corixa Corporation (Seattle, Wash.; see, for example, U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352, 1996. Another preferred adjuvant comprises a saponin, such as Quil A, or derivatives thereof, including QS21 and QS7 (Aquila Biopharmaceuticals Inc., Framingham, Mass.); Escin; Digitonin; or Gypsophila or Chenopodium quinoa saponins.

The present invention also provides methods of treating or preventing IL-13 mediated disease, any symptoms or diseases associated therewith, comprising administering an effective amount of a protein, a polynucleotide, a vector or a pharmaceutical composition according to the invention. Administration of a pharmaceutical composition may take the form of one or more individual doses, for example in a “prime-boost” therapeutic vaccination regime. In certain cases the “prime” vaccination may be via particle mediated DNA delivery of a polynucleotide according to the present invention, preferably incorporated into a plasmid-derived vector and the “boost” by administration of a recombinant viral vector comprising the same polynucleotide sequence, or boosting with the protein in adjuvant. Conversly the priming may be with the viral vector or with a protein formulation typically a protein formulated in adjuvant and the boost with a DNA vaccine of the present invention.

For the treatment of self-antigen, for example IL-13, mediated disease it is preferred that the adjuvant is a preferable inducer of a TH-1 response. In particular, the adjuvant comprises an immunostimulatory CpG oligonucleotide, such as disclosed in (WO96102555). Typical immunostimulatory oligonucleotides will be between 8-100 bases in length and comprises the general formula X₁ CpGX₂ where X₁ and X₂ are nucleotide bases, and the C and G are unmethylated.

The preferred oligonucleotides for use in adjuvants or vaccines of the present invention preferably contain two or more dinucleotide CpG motifs preferably separated by at least three, more preferably at least six or more nucleotides. The oligonucleotides of the present invention are typically deoxynucleotides. In a preferred embodiment the intemucleotide in the oligonucleotide is phosphorodithioate, or more preferably a phosphorothioate bond, although phosphodiester and other intemucleotide bonds are within the scope of the invention including oligonucleotides with mixed internucleotide linkages. e.g. mixed phosphorothioate/phophodiesters. Other internucleotide bonds which stabilise the oligonucleotide may be used. Methods for producing phosphorothioate oligonucleotides or phosphorodithioate are described in U.S. Pat. No. 5,666,153, U.S. Pat No. 5,278,302 and WO95/26204.

Examples of preferred oligonucleotides have the following sequences. The sequences preferably contain phosphorothioate modified internucleotide linkages.

| | | | |
|---|---|---|---|
| OLIGO 1: | TCC ATG ACG TTC CTG ACG TT | (CpG 1826) | (SEQ ID NO 5) |
| OLIGO 2: | TCT CCC AUC GTG CGC CAT | (CpG 1758) | (SEQ ID NO 6) |
| OLIGO 3: | ACC GAT GAC GTC GCC GGT GAC GGC ACC ACG | | (SEQ ID NO 7) |
| OLIGO 4: | TCG TCG TTT TGT CGT TTT GTC GTT | (CpG 2006) | (SEQ ID NO 8) |
| OLIGO 5: | TCC ATG ACG TTC CTG ATG CT | (CpG 1668) | (SEQ ID NO 9) |

Alternative CpG oligonucleotides may comprise the preferred sequences above in that they have inconsequential deletions or additions thereto.

The CpG oligonucleotides utilised in the present invention may be synthesized by any method known in the art (eg EP 468520). Conveniently, such oligonucleotides may be synthesized utilising an automated synthesizer. An adjuvant formulation containing CpG oligonucleotide can be purchased from Qiagen under the trade name “ImmunEasy”.

The compositions of the present invention may be used for both prophylaxis and therapy. The present invention provides a polypeptide or a polynucleotide according to the invention for use in medicine. The invention further provides the use of a polypeptide or a polynucleotide of the invention in the manufacture of a medicament for the treatment of allergies, respiratory ailments such as asthma and COPD, helminth-infection related disorders, fibrosis or cirrhosis of the liver.

The present invention also provides a method of vaccinating which comprises administering an effective amount of a vaccine composition of the invention to a patient and provoking an immune response to the vaccine composition.

The present invention also provides vaccine compositions as described herein for use in vaccination of a mammal against IL-13 mediated disorders such as allergies, respiratory ailments, helminth-infection related disorders, fibrosis and cirrhosis of the liver. Respiratory ailments include, for example, asthma, such as allergic asthma, and chronic obstructive pulmonary disease (COPD). Specifically, a vaccine composition capable of directing a neutralising response to IL-13 would therefore constitute a useful therapeutic for the treatment of asthma, particularly allergic asthma, in humans. It would also have application in the treatment of certain helminth infection-related disorders (Brombacher, 2000 Bioessays 22:646-656) and diseases where IL-13 production is implicated in fibrosis (Chiaramonte et al, 1999, J Clin Inv 104:777-785), such as chronic obstructive pulmonary disease (COPD) and cirrhosis of the liver.

The present invention provides a method of treatment of asthma or COPD by administering to an individual a vaccine composition that raises an immune response against IL-13 in said individual. IL-13 is a self-protein and accordingly the present inventors have produced chimaeric IL-13 antigens to render this self-protein immunogenic. It will be apparent to the man skilled in the art that other techniques may be used to render IL-13 immunogenic in a self-host, such as fusion or conjugation to carrier proteins and/or use of strong adjuvants. Accordingly, the present invention provides a method of treatment of asthma or COPD by vaccination of a host species with an immunogen that contains a portion of an autologous IL-13 sequence that has been rendered immunogenic for that host species.

Accordingly, there is provided a vaccine or immunogenic composition for the treatment of asthma in a host species, comprising an IL-13 immunogen wherein the IL-13 has been rendered immunogenic in that host, and an adjuvant.

The vaccine compositions of the invention may be administered in a variety of manners for example via the mucosal, such as oral and nasal; pulmonary, intramuscular, subcutaneous or intradermal routes. Where the antigen is to be administered as a protein based vaccine, the vaccine will typically be formulated with an adjuvant and may be lyophilised and resuspended in water for injection prior to use. Such compositions may be administered to an individual as an injectable composition, for example as a sterile aqueous dispersion, preferably isotonic. Typically such compositions will be administered intra muscularly, but other routes of administration are possible.

One technique for intradermally administration involves particle bombardment (which is also known as ‘gene gun’ technology and is described in U.S. Pat. No. 5,371,015). Proteins may be formulated with sugars to form small particles or DNA encoding the antigen may be coated on to inert particles (such as gold beads) and are accelerated at speeds sufficient to enable them to penetrate a surface of a recipient (e.g. skin), for example by means of discharge under high pressure from a projecting device. (Particles coated with nucleic acid vaccine constructs of the invention and protein sugar particles are within the scope of the present invention, as are devices loaded with such particles.) Other methods of administering the nucleic acid constructs or compositions containing said constructs directly to a recipient include ultrasound, electrical stimulation, electroporation and microseeding which is described in U.S. Pat. No. 5,697,901.

A nucleic acid construct of the present invention may also be administered by means of specialised delivery vectors useful in gene therapy. Gene therapy approaches are discussed for example by Verme et al, Nature 1997, 389:239-242. Both viral and non-viral systems can be used. Viral based systems include retroviral, lentiviral, adenoviral, adeno-associated viral, herpes viral and vaccinia-viral based systems. Non-viral based systems include direct administration of nucleic acids and liposome-based systems. For example, the vectors may be encapsulated by liposomes or within polylactide co-glycolide (PLG) particles.

A nucleic acid construct of the present invention may also be administered by means of transformed host cells. Such cells include cells harvested from a subject. The nucleic acid vaccine construct can be introduced into such cells in vitro and the transformed cells can later be returned to the subject. The nucleic acid construct of the invention may integrate into nucleic acid already present in a cell by homologous recombination events. A transformed cell may, if desired, be grown up in vitro and one or more of the resultant cells may be used in the present invention. Cells can be provided at an appropriate site in a patient by known surgical or microsurgical techniques (e.g. grafting, micro-injection, etc.). Suitable cells include dendritic cells.

The amount of vaccine composition which is delivered will vary significantly, depending upon the species and weight of mammal being immunised, the nature of the disease state being treated/protected against, the vaccination protocol adopted (i.e. single administration versus repeated doses), the route of administration and the potency and dose of the adjuvant compound chosen. Based upon these variables, a medical or veterinary practitioner will readily be able to determine the appropriate dosage level but it may be, for example, when the vaccine is a nucleic acid that the dose will be 0.5-5 μg/kg of the nucleic acid constructs or composition containing them. In particular, the dose will vary depending on the route of administration. For example, when using intradermal administration on gold beads, the total dosage will preferably between 1 μg-10 ng, particularly preferably, the total dosage will be between 10 μg and 1 ng. When the nucleic acid construct is administered directly, the total dosage is generally higher, for example between 50 μg and 1 or more milligram. The above dosages are exemplary of the average case.

In a protein vaccine, the amount of protein in each vaccine dose is selected as an amount which induces an immunoprotective response without significant, adverse side effects in typical vaccinees. Such amount will vary depending upon which specific immunogen is employed and how it is presented. Generally, it is expected that each dose will comprise 1-1000 μg of protein, preferably 1-500 μg, preferably 1-100 μg, most preferably 1 to 50 μg. An optimal amount for a particular vaccine can be ascertained by standard studies involving observation of appropriate immune responses in vaccinated subjects. Following an initial vaccination, subjects may receive one or several booster immunisation adequately spaced. Such a vaccine formulation may be either a priming or boosting vaccination regime; be administered systemically, for example via the transdermal, subcutaneous or intramuscular routes or applied to a mucosal surface via, for example, intra nasal or oral routes.

There can, of course, be individual instances where higher or lower dosage ranges are merited, and such are within the scope of this invention.

It is possible for the vaccine composition to be administered on a once off basis or to be administered repeatedly, for example, between 1 and 7 times, preferably between 1 and 4 times, at intervals between about 1 day and about 18 months, preferably one month. This may be optionally followed by dosing at regular intervals of between 1 and 12 months for a period up to the remainder of the patient's life. In an embodiment the patient will receive the antigen in different forms in a prime boost regime. Thus for example an antigen will be first administered as a DNA based vaccine and then subsequently administered as a protein adjuvant base formulation. Once again, however, this treatment regime will be significantly varied depending upon the size and species of animal concerned, the amount of nucleic acid vaccine and/or protein composition administered, the route of administration, the potency and dose of any adjuvant compounds used and other factors which would be apparent to a skilled veterinary or medical practitioner.

The following example illustrates the theory of the invention in mice rather than in humans, so that the protein is murine with mutations characteristic of human protein, but the results can readily be extrapolated to treatment of humans where the protein will have B cell epitopes from Human with mutations characteristic of a mouse, or other analogous protein.

Throughout the following examples of the invention, use is made of various widely known and practised techniques in molecular and cellular biology. Practical details of these may be found in a number of textbooks including Sambrook et al (1989, 2^{nd} edition. Cold Spring Harbor Press: New York). Amino acid sequences or designations may be given in either the one letter code, or the three letter code. The prefix ‘h’ is used to denote a protein or gene of human origin, ‘m’, murine origin and ‘c’, a chimaeric construct. ‘r’ is used to indicate a recombinant protein.

### EXAMPLES

1. Design of a Vaccine Against Murine IL-13

IL-13 belongs to the SCOP (Murzin et al, 1995, J Mol Biol 247:536-540) defined 4-helical cytokines fold family. Individual members of this fold superfamily are related structurally, but are difficult to align at the sequence level. The 3D structure of IL-13 has not yet been determined, but structures have been generated for a number of other 4-helical cytokines. Protein multiple sequence alignments were generated for IL-13 orthologues, and also for a number of other cytokines exhibiting this fold where the structure of at least one member had been determined (IL-4, GM-CSF, IL-5 and IL-2). Secondary structure predictions were performed for the IL-13 protein multiple sequence alignment using DSC (King and Sternberg, 1996, Prot Sci 5:2298-2310), SIMPA96 (Levin, 1997, Prot Eng 7:771-776) and Pred2ary (Chandonia and Karplus, 1995, Prot Sci 4:275-285). The individual cytokine protein multiple sequence alignments were aligned to each other, using both the sequence information and the structural information (from the known crystal structures and from the secondary structure prediction).

Antigenic sites, specifically B-cell epitopes, were predicted for murine IL-13 using the Cameleon software (Oxford Molecular), and these were mapped onto the IL-4 structure (accession number 1RCB in the Brookhaven database) using the protein multiple sequence alignment to give an idea of where they might be located structurally on IL-13. From this analysis, exposed regions which were potentially both antigenic and involved in receptor binding were selected.

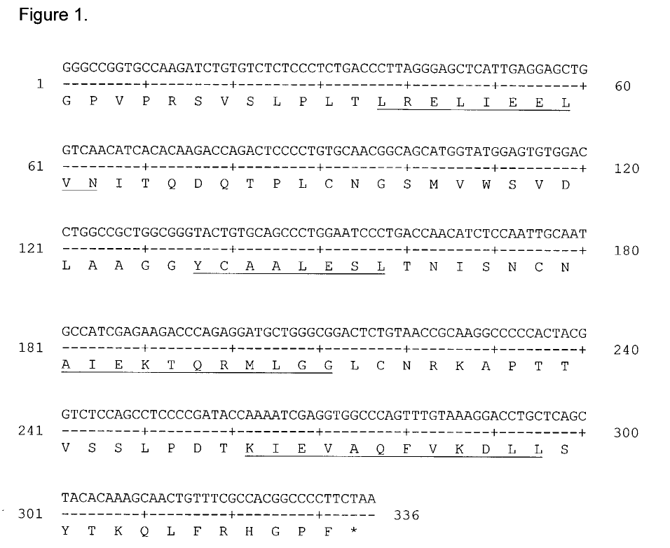

From this model, a chimaeric IL-13 sequence was designed in which the sequence of the predicted antigenic loops was taken from murine IL-13, and the sequence of the predicted structural (predominantly helical) regions was taken from human IL-13. The purpose of this design was to identify target epitopes from murine IL-13 against which neutralising antibodies might be raised, and to present them on a framework which was structurally similar to the native protein, but yet contained sufficient sequence variation to the native (murine) protein to ensure that one or more CD4 T helper epitopes would be present. The nucleic acid and protein sequences selected for this example of a chimaeric IL-13 vaccine are shown in FIG. 1 (SEQ ID NO 19 and 20). The underlined sequences correspond to sequences found in the human orthologue. Twelve amino acids were substituted to achieve the sequence in FIG. 1. It should be understood that the degeneracy of the genetic code allows many possible nucleic acid sequences to encode identical proteins. Furthermore, it will be appreciated that there are other possible chimaeric IL-13 vaccine designs within the scope of the invention, that have other orthologus mutations in non-exposed areas.

1.2 Preparation of Chimaeric IL-13

Chimaeric IL-13 (cIL-13) DNA sequence was synthesised from a series of partially overlapping DNA oligonucleotides, with the sequences cIL-13-1 to cIL-13-6 shown in Table 1. These oligos were annealed, and cIL-13 DNA generated by a PCR with the cycle specification of 94° C. for 1 minute followed by 25 cycles of 94° C. for 30 seconds, 55° C. for 1 minute and 72° C. for 2minutes. Followed by 72° C. for 7 minutes and cooling to 4° C. when finished. The reaction product comprised a band of the expected size, 361 base pairs, which was subcloned into the T/A cloning vector pCR2.1 (Invitrogen, Groningen, Netherlands) to generate pCR2.1-cIL-13. A BamH1 and Xho1 cIL-13 digested fragment from pCR2.1-cIL-13 was then subcloned into the BamH1 and Xho1 sites in pGEX4T3 (Amersham Pharmacia, Amersham, Bucks, UK) generating pGEX4T3-cIL-13/1. On sequencing the pGEX4T3-cIL-13/1 construct we discovered an extra 39 base pairs of DNA sequence (derived from the pCR2.1 vector) between the sequence for GST and cIL-13. To correct this, we repeated the PCR for cIL-13 using pGEX4T3-cIL-13/1 and primers cIL-13Fnew and cIL-13R. The PCR product obtained was then cloned back into pGEX4T3 using BamH1 and Xho1 restriction sites, to generate the expression vector pGEX4T3-cIL-13. The sequence of this construct was verified by dideoxy terminator sequencing. This vector encodes a genetic fusion protein consisting of glutathione-S-transferase and cIL-13 (GST-cIL-13). The two moieties of the protein are linked by a short spacer which contains the recognition site for thrombin. The fusion protein may be readily purified by glutathione sepharose affinity chromatography, and then used directly, or a preparation of free cIL-13 produced by cleavage with thrombin.

TABLE 1
Oligonucleotides used to construct chimaeric
IL-13.

| Oligo | Sequence (5′-3′) |
|---|---|
| cIL-13-1R (SEQ | TGTGATGTTGACCAGCTCCTCAATGAGCTCCCTAAG |
| ID NO 10) | GGTCAGAGGGAGAGACACAGATCTTGGCACCGGCCC |
| cIL-13-2F (SEQ | AGGAGCTGGTCAACATCACACAAGACCAGACTCCCC |
| ID NO 11) | TGTGCAACGGCAGCATGGTATGGAGTGTGGACCTGG |
| | C |
| cIL-13-3R (SEQ | GCAATTGGAGATGTTGGTCAGGGATTCCAGGGCTGC |
| ID NO 12) | ACAGTACCCGCCAGCGGCCAGGTCCACACTCCATAC |
| cIL-13-4F (SEQ | TGACCAACATCTCCAATTGCAATGCCATCGAGAAGA |
| ID NO 13) | CCCAGAGGATGCTGGGCGGACTCTGTAACCGCAAGG |
| | C |
| cIL-13-5R (SEQ | AAACTGGGCCACCTCGATTTTGGTATCGGGGAGGCT |
| ID NO 14) | GGAGACCGTAGTGGGGGCCTTGCGGTTACAGAGTCC |
| cIL-13-6F | AAATCGAGGTGGCCCAGTTTGTAAAGGACCTGCTCA |
| (SEQ ID NO 15) | GCTACACAAAGCAACTGTTTCGCCACGGCCCCTTC |
| cIL-13F (SEQ | CGCGGATTCGGGCCGGTGCCAAGATCTG |
| ID NO 16) | |
| cIL-13R (SEQ | CTCCGCTCGAGTCGACTTAGAAGGGGCCGTGGCGAA |
| ID NO 17) | A |
| cIL-13Fnew | CGCGGATCCGGGCCGGTGCCAAGATCTG |
| (SEQ ID NO 18) | |

The pGEX4T3-cIL-13 expression vector was transformed into E.coli BLR strain (Novagen, supplied by Cambridge Bioscience, Cambridge, UK). Expression of GST-cIL-13 was induced by adding 0.5 mM IPTG to a culture in the logarithmic growth phase for 4 hrs at 37° C. The bacteria were then harvested by centrifugation and GST-cIL-13 purified from them by a method previously described for purification of a similar GST-human IL-13 fusion protein (McKenzie et al, 1993, Proc Natn Acad Sci 90:3735-3739).

Characterisation of cIL-13 Properties

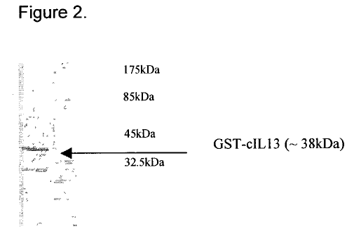

Samples of purified GST-cIL-13 were analysed by SDS-PAGE electrophoresis. FIG. 2 shows that the purified preparation contains a protein of the expected size for GST-cIL-13. The lower band represents a small quantity of GST, arising due to partial cleavage of the fusion protein during preparation.

To confirm that the purified protein was GST-cIL-13, samples were separated by SDS-PAGE, blotted onto PVDF membrane and then analysed for the presence of IL-13 and GST immunoreactivity by Western blotting. Since cIL-13 contains sequence arising from both human and murine IL-13, it was expected that it would be recognised by specific antisera directed at human IL-13 or mouse IL-13. Blots were blocked with 3% bovine serum albumin (BSA) in TBS (50 mM trizma hydrochloride, 138 mM sodium chloride, 2.7 mM potassium chloride, pH8.0) containing 0.05% Tween-20 (TBST) overnight at 4° C., incubated with primary antibody for 1 hour at room temperature (RT) with shaking then washed 4 times with TBST. Secondary antibody was added for 1 hour at RT with shaking, prior to washing 4 times and developing with SuperSignal Chemiluminescent Reagent (Pierce, Rockford, Ill., USA).

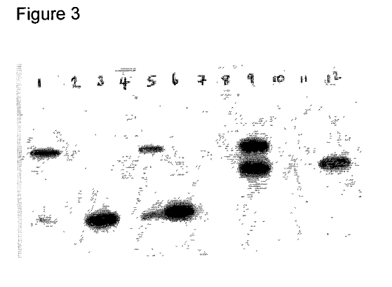

FIG. 3 (legend below) illustrates the results of this analysis, which indicate that the purified protein is recognised by antibodies to human IL-13, mouse IL-13 and GST, so confirming the expected structure.

| Lane | Sample | Primary Antibody |
|---|---|---|
| 1 | GST-cIL-13 | Anti-mIL-13 |
| 2 | rhIL-13 | Anti-mIL-13 |
| 3 | rmIL-13 | Anti-mIL-13 |
| 4 | Markers | — |
| 5 | GST-cIL-13 | Anti-hIL-13 |
| 6 | rhIL-13 | Anti-hIL-13 |
| 7 | rmIL-13 | Anti-hIL-13 |
| 8 | Markers | — |
| 9 | GST-cIL-13 | Anti-GST |
| 10 | rhIL-13 | Anti-GST |
| 11 | rmIL-13 | Anti-GST |
| 12 | GST | Anti-GST |

The primary antibodies used in this experiment were: anti-hIL-13, catalogue number AF-213-NA, R&D Systems, Abingdon, Oxford, UK, used at 1 μg/ml; anti-mIL-13, catalogue number AF-413-NA, R&D Systems, used at 1 μg/ml and anti-GST, catalogue number 27-4590D, Pharmacia, used at 1/200. The secondary antibodies used in this experiment were: HRP-conjugated anti-goat IgG, catalogue number A-5420, Sigma-Aldrich Company Ltd, Poole, Dorset, UK, used at 1/40,000.

The protein samples were GST-cIL-13, prepared as described in Example 2, recombinant human IL-13 (rhIL-13), catalogue number CH1-013, Cambridge Bioscience, Cambridge, UK, recombinant mouse IL-13 (rmIL-13) catalogue number 413-ML-025, R&D Systems, and GST, prepared from E.coli transfected with empty pGEX4T3 vector as described (Sambrook et al, 1989, 2^{nd} edition. Cold Spring Harbor Press: New York).

1.3 Conformation of Chimaeric IL-13

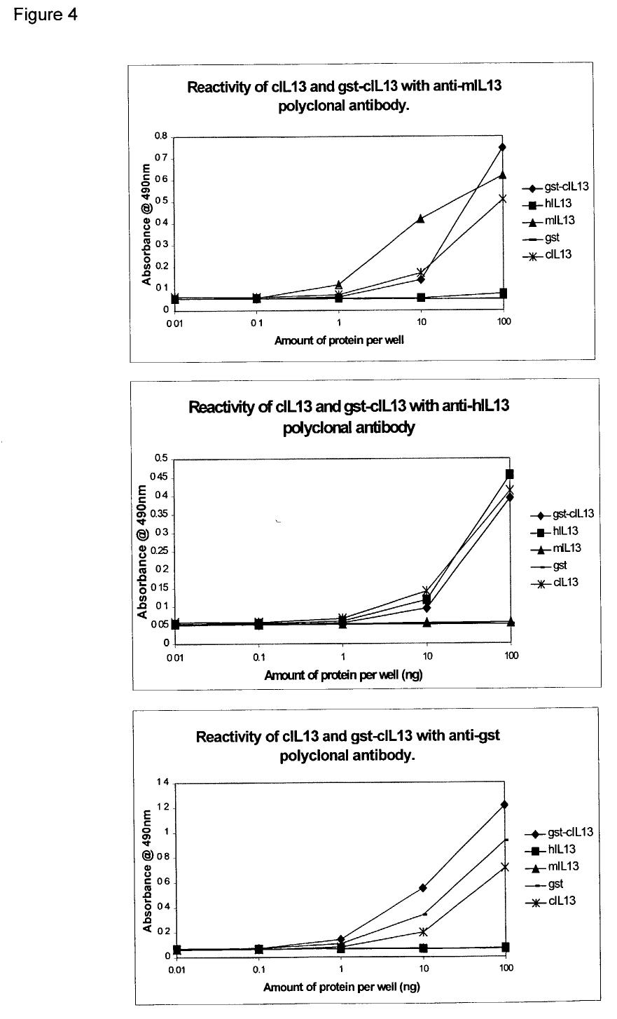

To confirm that GST-cIL-13 adopts a similar conformation in solution to that of native IL-13, samples of GST-cIL-13 and cIL-13 (generated from GST-cIL-13 by thrombin cleavage) were analysed by ELISA. 96-well Maxisorp plates (Life Technologies Ltd, Paisley, UK) were coated with cIL-13, GST-cIL-13, mIL-13, hIL-13 or gst in carbonate-bicarbonate buffer, overnight at 4° C. Plates were then blocked with 3% BSA/TBST for 1 hour at RT, washed 3 times in TBST, incubated with primary antibody for 1 hour at RT then washed 3 times in TBST. Secondary antibody was added for 1 hour, washed 3 times in TBST, then developed with 0-phenylenediamine dihydrochloride peroxidase substrate (OPD, Sigma Aldrich) for 30 minutes. The primary and secondary antibodies used in this experiment were as described above. As shown in FIG. 4, GST-cIL-3 and cIL-13 were specifically recognised by antibodies to human IL-13 and mouse IL-13. These data confirm that the chimaerisation process has not grossly altered the protein confirmation.

1.4 Binding of Chimaeric IL-13 to Receptors

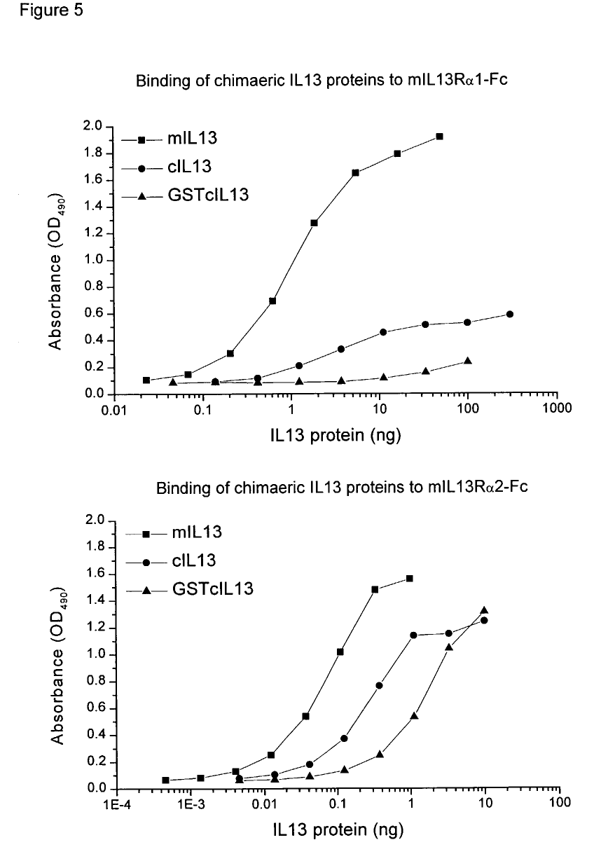

ELISAs were set up to determine whether cIL-13 could bind to either of the known mouse IL-13 receptors (mIL-13R1 or mIL-13R2). 96-well Maxisorp plates were coated with anti-human IgG (catalogue number I-3382, Sigma Aldrich) in carbonate-bicarbonate buffer overnight at 4° C. Plates were then blocked with 3% BSA/TBST for 1 hour at RT, washed 3 times in TBST, and incubated with mIL-13R1-Fc or mIL-13R2-Fc (catalogue numbers 491-IR-200 and 539-IR-100 respectively, R+D Systems) for 1 hour at RT. After washing, plates were incubated with dilutions of mIL-13 or cIL-13 or GST-cIL-13 for 1 hour at RT, washed again and incubated with biotinylated anti-mIL-13 (catalogue number BAF413, R+D Systems). Following further washing and incubation with streptavidin conjugated horse-radish peroxidase, the plates were developed with 0-phenylenediamine dihydrochloride peroxidase substrate for 30 minutes. As shown in FIG. 5, cIL-13 and GST-cIL-13 are both able to bind to either of the mIL-13 receptors. Again, these data confirm that the chimaerisation process has not grossly altered the protein confirmation.

1.5 Bioactivity of Chimaeric IL-13

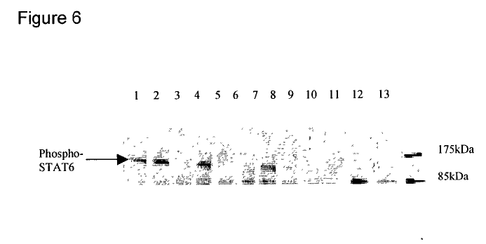

The bioactivity of GST-cIL-13 was assessed by the ability of this protein to phosphorylate STAT6 in the human lung fibroblast cell line A549. These cells express the human type-2 IL-4 receptor that is responsive to both IL-4 and IL-13. Stimulation of these cells with hIL-4, hIL-13 or mIL-13 induces phosphorylation of the signalling protein STAT6. 5×10⁵ A549 cells were plated into 60 mm tissue culture dishes (Life Technologies) in RPMI (Life Technologies) and grown to 70% confluence. Cells were then incubated with between 2 and 150 ng/ml cytokine or purified cIL-13 for 15 mins at 37° C. Because the presence of a GST fusion partner may alter the bioactivity of cytokines, the chimaeric IL-13 was assayed as both GST-cIL-13 fusion protein, and free cIL-13 liberated from the fusion by thrombin cleavage. By way of control, rmIL-13 and GST were also tested. Cell lysates were then prepared and analysed by Western blot for the presence of phospho-STAT6 using rabbit anti-phospho-STAT6 polyclonal antibody (NEB, Hitchin, Herts, UK. Catalogue number 9361S). Blots were blocked overnight in 5% BSA/TBST (BSA must be A-7906 from Sigma as primary antibody is phospho-specific, 0.1% Tween-20), primary antibody was added at 1/1000 for 1 hour at RT then washed 3 times with TBST. Anti-rabbit HRP conjugated secondary antibody (A-4914, Sigma Aldrich) was added at 1/5000 for 1 hour at RT then washed 4 times with TBST prior to developing with the HRP chemiluminescent substrate ECL Reagent (Amersham Pharmacia). The results of this experiment are shown in FIG. 6.

Each lane was loaded with the following protein:

| Lane | Lysates of A549 cells treated with . . . | |
|---|---|---|
| 1 | 50 | ng/ml rmIL-13 |
| | (R&D Systems) | |
| 2 | 10 | ng/ml rmIL-13 |
| | (R&D Systems) | |
| 3 | 2 | ng/ml rmIL-13 |
| | (R&D Systems) | |
| 4 | 50 | ng/ml cIL-13 |
| 5 | 10 | ng/ml cIL-13 |
| 6 | 2 | ng/ml cIL-13 |
| 7 | 150 | ng/ml GST-cIL-13 |
| 8 | 30 | ng/ml GST-cIL-13 |
| 9 | 6 | ng/ml GST-cIL-13 |
| 10 | No treatment | |
| 11 | 1 | μg/ml GST |
| 12 | 0.25 | μg/ml GST |

| | |
|---|---|
| 13 | Molecular weight markers |

Recombinant protein reagents were as described in FIG. 3.

Treatment of A549 cells with 50 or 10 ng/ml (but not 2 ng/ml) rmIL-13 induced the phosphorylation of STAT6, indicating bioactivity. Treatment of A549 cells with 50 ng/ml (but not 10 or 2 ng/ml) cIL-13 induced the phosphorylation of STAT6, indicating bioactivity. Similarly, 150 ng/ml GST-cIL-13 (which is approximately equivalent in molar terms to 50 ng/ml cIL-13) is bioactive, whereas 30 and 6 ng/ml are not. CIL-13 is therefore an agonist at this receptor, but under these experimental conditions approximately 5 fold less bioactive than mIL-13.

1.6 Immunisation with cIL-13

cIL-13 and GST-cIL-13 were then used as immunogens to induce the formation of auto-antibodies against mouse IL-13 in Balb/c mice. Female mice aged 6-8weeks were given one subcutaneous (sc) injection of approximately 30 μg protein in complete Freunds adjuvant (CFA) at the base of the tail. This was followed by three booster immunisations at the same site, each consisting of approximately 10 μg protein in incomplete Freunds adjuvant [IFA] for boosts. Each treatment group contained 5 animals, and they were immunised according to the protocol in Table 2.

TABLE 2

| Group | Immunisation |
|---|---|
| A | Saline control in CFA/IFA s/c |
| B | 30/10 μg GST in CFA/IFA s/c |
| C | Non immunised naïve mice |
| D | 30/10 μg GST-hIL-13 in CFA/IFA s/c |
| E | 30/10 μg cIL-13 in CFA/IFA s/c |
| F | 30/10 μg GST-cIL-13 in CFA/IFA s/c |
| Day | Treatment |
| −12 | Pre-bleed |
| 0 | Primary immunisation |
| 14 | 1^{st} Boost Immunisation |
| 27 | Tail bleed |
| 42 | Tail bleed |
| 49 | 2^{nd} Boost Immunisation |
| 70 | Tail bleed |
| 97 | Tail bleed |
| 99 | 3^{rd} Boost Immunisation |
| 113 | Tail bleed |
| 140 | Tail bleed |

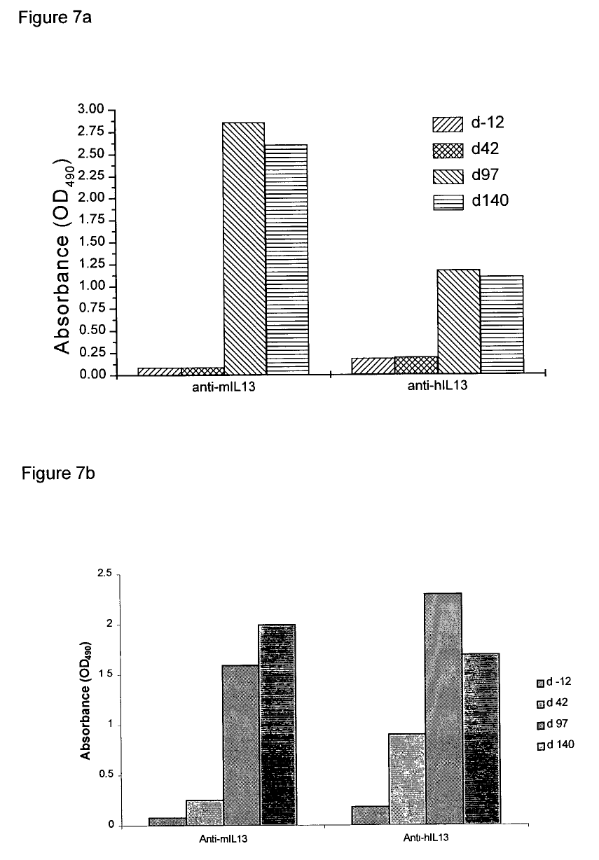

Serum samples were obtained by venepuncture of the tail vein at the timepoints specified in Table 2. After clarification by centrifugation, the samples were assayed by ELISA for the presence of specific IgG responses to mouse IL-13, human IL-13 and GST. None of the animals in groups A-D possessed anti-mouse IL-13 antibodies at any time point. All of the animals in groups B, D and F made a strong IgG response to GST (group E animals also made strong antibody responses to GST, because there was GST remaining in the cIL-13 sample used to immunise these mice). Anti-mouse IL-13 antibody responses were induced in five out of five animals in group F and four out of five animals in group E. FIG. 7 (a and b) shows the serological analysis for one of these animals in group F and one of these animals from group E 7b (gst-cIL-13 immunised and cIL-13 immunised respectively). The results indicate that immunisation with GST-cIL-13 or cIL-13 was able to break tolerance to mIL-13, generating mouse anti-mIL-13 antibodies.

Sera from two mice (F1d70 and F5d97) that had strong anti-mIL-13 IgG responses, were tested for the capacity to neutralise the bioactivity of rmIL-13 in the A549/phospho-STAT6 assay. 20 ng/ml or 10 ng/ml rmIL-13 (R&D Systems) were incubated with 1% sera in serum free RPMI tissue culture media for 15 minutes at room temperature prior to a 15 minute incubation at 37° C. with A549 cells. Cell lysates were prepared and analysed by Western blot for the presence of phospho-STAT6 as previously described above. As a negative control, anti-hIL-13 serum was obtained from a Balb/c mouse immunised with GST-hlL-13 and shown by ELISA to have a strong anti-hIL-13 IgG response, but no anti-mIL-13 antibodies. As a positive control, normal mouse serum was spiked with a neutralising anti-mIL-13 antibody (R&D Systems, catalogue number AF-413-NA) to give a final concentration of 1 μg/ml.

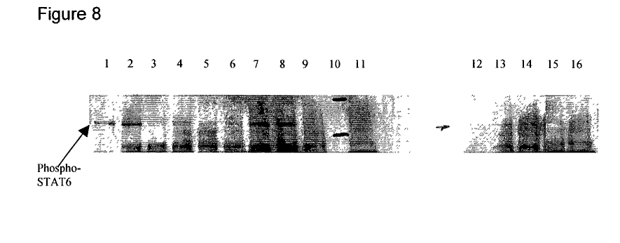

The results of this experiment are shown in FIG. 8, in which the following was tested:

| Lane | Cytokine | | Antibody |
|---|---|---|---|
| 1 | 20 | ng/ml rmIL-13 | Normal mouse serum |
| 2 | 10 | ng/ml rmIL-13 | Normal mouse serum |
| 3 | 0 | ng/ml rmIL-13 | Normal mouse serum |
| 4 | 20 | ng/ml rmIL-13 | Serum sample F1d70 |
| 5 | 10 | ng/ml rmIL-13 | Serum sample F1d70 |
| 6 | 0 | ng/ml rmIL-13 | Serum sample F1d70 |
| 7 | 20 | ng/ml rmIL-13 | Anti-hIL-13 mouse serum |
| 8 | 10 | ng/ml rmIL-13 | Anti-hIL-13 mouse serum |
| 9 | 0 | ng/ml rmIL-13 | Anti-hIL-13 mouse serum |
| 10 | Molecular weight markers | | — |
| 11 | 0 | ng/ml rmIL-13 | Normal mouse serum + anti-mIL-13 |
| 12 | 20 | ng/ml rmIL-13 | Serum sample F5d97 |
| 13 | 10 | ng/ml rmIL-13 | Serum sample F5d97 |
| 14 | 0 | ng/ml rmIL-13 | Serum sample F5d97 |
| 15 | 20 | ng/ml rmIL-13 | Normal mouse serum + anti-mIL-13 |
| 16 | 10 | ng/ml rmIL-13 | Normal mouse serum + anti-mIL-13 |

Immunisation with a chimaeric IL-13 immunogen of the invention induces the production of auto-antibodies against mouse IL-13, capable of neutralising the biological activity of the mouse IL-13 (lanes 4, 5, 12, 13), in a fashion comparable to exogenously added anti-murine IL-13 antibody (lanes 15, 16). This activity is not present in normal mouse serum (lanes 1,2), nor in serum from animals immunised with GST-hIL-13 (lanes 7, 8).

These data provide a basis for treating mammals with an IL-13 dependent pathology by vaccinating them with cIL-13, and so inducing an endogenous neutralising antibody activity.

1.7 Alternative Constructs

1.7.1 6 his Tagged cIL-13 Design.

GST-cIL-13 is bacterially produced protein is insoluble and requires solubilisation and refolding in vitro. Size exclusion chromatography indicates that the refolding process generates several differentially folded forms, which suggest that a proportion of the immune response is being directed against forms that may be generating irrelevant antibodies that do not bind native mouse IL-13.

Therefore this candidate may not be generating the most potent neutralising anti-mouse IL-13 antibody responses possible.

For this reason 6 his-cIL-13 has been cloned into a mammalian expression vector, mammalian expressed 6 his-cIL-13 is soluble and does not require refolding in vitro.

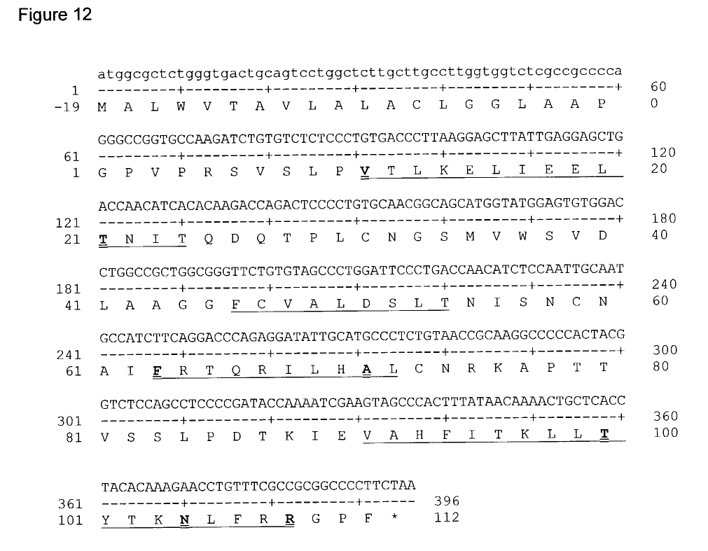

1.7.2 FIG. 12 (SEQ ID NO 23 and 24) shows a vaccine antigen where different analogous mutations are made. Protein sequence numbering according to a scheme where the glycine residue in the sequence “GPVPR” is residue 1. Single underlined sequences correspond to the predicted helical regions from the revised structural model. Double underlined bold residues indicate points at which mutations are incorporated into the mouse sequence:

| | |
|---|---|
| 11 | mouse Leu changed to Val (rat) |
| 21 | mouse Ser changed to Thr (non-orthologous) |
| 63 | mouse Tyr changed to Phe (non-orthologous) |
| 71 | mouse Gly changed to Ala (dog/pig/cow) |
| 100 | mouse Ser changed to Thr (dog) |
| 104 | mouse Gln changed to Asn (non-orthologous) |
| 108 | mouse His changed to Arg (non-orthologous) |

1.8 Application to Human Therapy

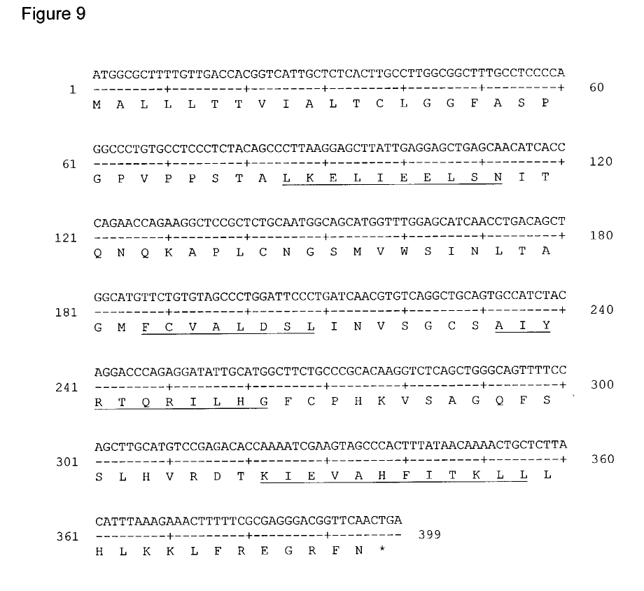

FIG. 9 shows one possible vaccine antigen according to the invention directed at the production of anti-human IL-13 antibodies in humans. This will be useful for the treatment of diseases characterised by excessive or inappropriate IL-13, for example asthma. The sequence corresponding to mouse IL-13 are underlined. The construct contains twelve amino-acid substitutes that are analogous to murine IL-13. These are:

| | | | | | |
|---|---|---|---|---|---|
| R | → | K | at | position | 30 |
| V | → | S | at | position | 37 |
| Y | → | F | at | position | 63 |
| A | → | V | at | position | 65 |
| E | → | D | at | position | 68 |
| E | → | Y | at | position | 80 |
| K | → | R | at | position | 81 |
| M | → | I | at | position | 85 |
| G | → | H | at | position | 87 |
| Q | → | H | at | position | 113 |
| V | → | I | at | position | 115 |
| D | → | K | at | position | 117 |

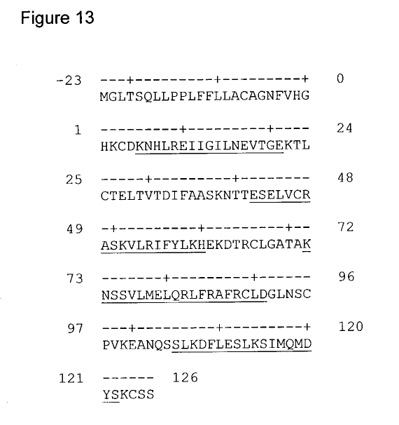

FIG. 13 (SEQ ID NO 25) shows one possible vaccine for human use based on Chimaeric IL-4. It is an Example of a chimearic human IL-4 vaccine protein. Underlined amino-acid residues comprise the alpha-helical structural regions and are derived from mouse IL-4 with the inclusion of amino acid 21 into the first helix. Plain symbols indicate amino-acid residues derived from human IL-4. Positions of the alpha-helical regions are taken from Zuegg, J et al (2001) Immunol and Cell Biol 79:332-339.

### Example 2

### Immune Response to gst-cIL-13 is Specific for Mouse IL-13 and does not Cross React with Mouse IL-4

As mouse IL-13 is structurally similar to mouse IL-4, sera from a GST-cIL-13 immunised mouse (that had been shown to contain high titre anti-mouse IL-13 autoantibodies) was analysed for cross-reactivity to mouse IL-4 using an anti-mouse IL-4 ELISA and an in vitro mIL-4 neutralisation bioassay.

2.1 Anti-mouse IL-4 ELISA.

96-well Maxisorp plates were coated with anti-mouse IL-4 monoclonal antibody (Cat. No. MAB404, R+D Systems) in carbonate-bicarbonate buffer overnight at 4° C. Plates were then blocked with 3% BSA/TBST for 1 hour at RT, washed 3 times in TBST, and incubated with mouse IL-4 (Cat. No. 404-ML-005, R+D Systems) for 1 hour at RT. After washing, plates were incubated with mouse sera for 1 hour at RT, washed again and incubated with HRP conjugated anti-mouse IgG polyclonal antibody (Cat. No. A-9309, SIGMA). Following further washing, the plates were developed with 0-phenylenediamine dihydrochloride peroxidase substrate for 30 minutes.

The level of anti-mouse IL-4 antibodies in the serum was expressed as an endpoint titre. The endpoint titre is defined as that dilution of serum that is equivalent to twice the ELISA background reading.

| | Anti-mouse IL-4 | Anti-mouse IL-13 |
|---|---|---|
| Mouse | antibody endpoint titre | antibody endpoint titre |
| C2 | 1/900 | 1/80000 |
| (serum sample taken at | | |
| day 125, post 4 × GST- | | |
| cIL-13 vaccine doses) | | |

A very low level of mouse IL-4 cross-reactivity was detected in this serum sample. In contrast, a much higher anti-mouse IL-13 antibody endpoint titre was previously determined in this serum sample, using an anti-mouse IL-13 antibody ELISA. The level of mouse IL-4 cross-reactivity determined by this ELISA, would not be expected to have mouse IL-4 neutralising effects in vivo. This serum sample was assessed for mouse IL-4 neutralisation capacity in an in vitro mouse IL-4 bioassay.

2.2 In vitro Mouse IL-4 Neutralisation Bioassay.

Mouse IL-4 stimulates the proliferation of CTLL cells in vitro. An assay was therefore developed in these cells, to assess the mouse IL-4 neutralisation capacity of serum from this GST-cIL-13 vaccinated mouse.

To measure the ability of mouse serum to neutralise the bioactivity of recombinant mouse IL-4 on mouse CTLL cells (Cat. No. 87031904, ECACC), 3 ng/ml recombinant mouse IL-4 was incubated with various concentrations of sera for 1 hour at 37° C. in a 96-well tissue culture plate (Invitrogen). Following this pre-incubation period, CTLL cells were added. The assay mixture, containing various serum dilutions, recombinant mouse IL-4 and CTLL cells, was incubated at 37° C. for 70 hours in a humidified CO₂ incubator. MTT substrate (Cat. No. G4000, Promega) was added during the final 4 hours of incubation, after which the reaction was stopped with an acid solution to solubilise the metabolised blue formazan product. The absorbance of the solution in each well was read in a 96-well plate reader at 570 nm wavelength.

Note that this assay is only able to measure mouse IL-4 neutralisation capacity in serum dilutions greater than or equivalent to 1/100. Serum dilutions less than 1/100 induce non-specific proliferative effects in CTLL cells.

The capacity of the serum to neutralise mouse IL-4 bioactivity was expressed as, that dilution of serum required to neutralise the bioactivity of a defined amount of mouse IL-4 by 50% (=ND₅₀). The more dilute serum sample required, the more potent the neutralisation capacity.

The highest concentration of mouse C2 serum tested was a 1/100 dilution. This did not neutralise the bioactivity of 3 ng /ml mouse IL-4 by 50%, therefore the ND₅₀ is expressed as <1/100 dilution.

| | Mouse IL-4 | Mouse IL-13 |
|---|---|---|
| | neutralisation capacity | neutralisation capacity |
| Mouse | (ND₅₀) | (ND₅₀) |
| C2 | <1/100 | 1/5300 |
| (serum sample taken at | | |
| day 125, post 4 × GST- | | |
| cIL-13 vaccine doses) | | |

No mouse IL-4 neutralisation capacity was detected in this serum sample at the dilutions of serum tested. In contrast (when assessed for mouse IL-13 neutralisation capacity), this serum sample potently neutralised mouse IL-13 bioactivity.

These data demonstrate that although a very low level of mouse IL-4 cross-reactivity can measured in the serum by an anti-mouse IL-4 antibody ELISA, there is no associated mouse IL-4 neutralisation capacity.

2.3 New Mouse IL-13 Neutralisation Bioassay to Assess the Mouse IL-13 Neutralisation Capacity of Mouse Serum Samples.

Previous GST-cIL-13 bioactivity and mouse IL-13 neutralisation capacity data were generated using a STAT-6 phosphorylation readout in A549 cells. This assay was cumbersome and not easily amenable for the generation of quantitative data. Mouse IL-13 stimulates the proliferation of TF-1 cells in vitro. An assay was therefore developed in these cells to assess the mouse IL-13 neutralisation capacity of serum from GST-cIL-13 vaccinated mice.

2.4 In vitro Mouse IL-13 Neutralisation Bioassay.

To measure the ability of mouse serum to neutralise the bioactivity of recombinant mouse IL-13 on human TF-1 cells (obtained in-house), 5 ng/ml recombinant mouse IL-13 was incubated with various concentrations of sera for 1 hour at 37° C. in a 96-well tissue culture plate (Invitrogen). Following this pre-incubation period, TF-1 cells were added. The assay mixture, containing various serum dilutions, recombinant mouse IL-13 and TF-1 cells, was incubated at 37° C. for 70 hours in a humidified CO₂ incubator. MTT substrate (Cat. No. G4000, Promega) was added during the final 4 hours of incubation, after which the reaction was stopped with an acid solution to solubilise the metabolised blue formazan product. The absorbance of the solution in each well was read in a 96-well plate reader at 570 nm wavelength.

Note that this assay is only able to measure mouse IL-13 neutralisation capacity in serum dilutions greater than or equivalent to 1/100. Serum dilutions less than 1/100 induce non-specific proliferative effects in TF-1 cells.

The capacity of the serum to neutralise mouse IL-13 bioactivity was expressed as, that dilution of serum required to neutralise the bioactivity of a defined amount of mouse IL-13 by 50% (=ND₅₀). The more dilute serum sample required, the more potent the neutralisation capacity.

The mouse IL-13 neutralisation capacity of serum from GST-cIL-13 immunised mice was measured by the above method. Potent IL-13 neutralising responses were generated, as indicated below.

| Mouse | |
|---|---|
| (Serum samples taken at | Mouse IL-13 |
| day 125, post 4 × GST- | neutralisation capacity |
| cIL-13 vaccine doses) | (ND₅₀) |
| C1 | 1/1250 |
| C2 | 1/5230 |
| C3 | 1/523 |
| C4 | 1/417 |
| C5 | 1/1670 |

2.5 Determination of the Level of Mouse IL-13 Neutralisation Required for Efficacy in the ‘Ovalburnin Challenge’ Mouse Asthma Model.

In order to benchmark the required potency of an IL-13 autovaccine for treatment of asthma, mice were treated with various doses of rabbit anti-mouse IL-13 polyclonal antibody (administered passively by intra-peritoneal injection) during ovalbumin challenge, in the ‘ovalbumin challenge’ mouse asthma model. Model parameters such as airway hyper-responsiveness (AHR), goblet cell metaplasia (GCM) and lung inflammatory cell content were measured at the end of this experiment. Efficacy in this model was correlated to the levels of mouse IL-13 neutralisation achieved in mouse serum. The mouse IL-13 neutralisation bioassay was used to determine the level of mouse IL-13 neutralisation in serum samples.

| Treatment group | | |
|---|---|---|
| (Dose of passively | | Mouse IL-13 |
| administered rabbit anti- | | neutralisation capacity |
| mouse IL-13 antibody) | | (ND₅₀) |
| Highest | dose | 1/4100 |
| High | dose | 1/2670 |
| Mid | dose | 1/476 |
| Lowest | dose | 1/207 |

Treatment groups given the highest three doses of antibody all performed similarly. All of these three groups showed efficacy equivalent to (for AHR) or better than (for GCM) the gold standard treatment (dexamethasone, administered by the intraperitoneal route at 3×1.5 mg/kg) used in this model. The ‘lowest dose’ of antibody administered, showed efficacy somewhere between that of dexamethasone and the ‘no treatment’ positive control groups.

Therefore the level of IL-13 neutralisation achieved in the ‘mid dose’ treatment group, represents the required potency threshold for an IL-13 autovaccine in this animal model. The potency threshold is defined as the lowest level of IL-13 neutralisation in mouse serum, required to show 100% efficacy in the asthma model (=ED₁₀₀). 1×ED₁₀₀ is therefore equivalent to an ND₅₀ of 1/476.

Significance of Defined Potency Threshold.

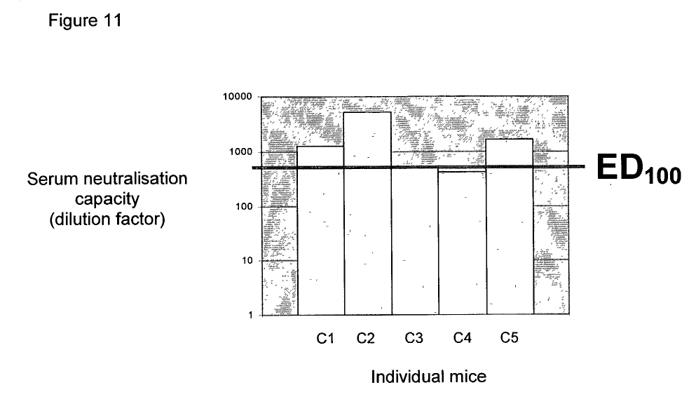

The level of IL-13 neutralisation required for efficacy in the ‘ovalbumin challenge’ mouse asthma model has been defined above. The levels of IL-13 neutralisation induced by GST-cIL-13 in mice C1-3 and C5, are in excess of the potency threshold required for efficacy in the asthma model. These results are illustrated in FIG. 11.

Therefore the GST-cIL-13 vaccine would be expected to show efficacy in the mouse asthma model.

### Example 3

### Immunogenicity Profile of GST-cIL-13 in Combination with Various Adjuvants

3.1 Immunisation Protocol.

GST-cIL-13 was used as an immunogen to induce the formation of auto-antibodies against mouse IL-13 in Balb/c mice. Female mice aged 6-8 weeks were given one injection of approximately 100 μg protein in adjuvant. This was followed by four booster immunisations each consisting of 50 μg protein in adjuvant (See below for immunogen+adjuvant formulations). Each treatment group contained 5 animals, immunised according to the protocol in the table below.

Serum samples were obtained by venepuncture of the tail vein at the timepoints specified. After clarification by centrifugation, the samples were assayed by ELISA for the presence of specific IgG responses to mouse IL-13.

| Group | Immunisation |
|---|---|
| A | GST-cIL-13 in AS03 i/m |
| B | GST-cIL-13 in Alum i/p |
| C | GST-cIL-13 in ‘ImmunEasy’ i/m |
| D | GST-cIL-13 in CFA/IFA s/c |
| E | GST-cIL-13 in PBS s/c |
| F | No immunisations |

| Day | Treatment |
|---|---|
| −7 | Pre-bleed |
| 0 | Primary immunisation |
| 21 | 1^{st} boost immunisation |
| 35 | Tail bleed |
| 49 | 2^{nd} boost immunisation |
| 63 | Tail bleed |
| 77 | 3^{rd} boost immunisation |
| 92 | Tail bleed |
| 106 | 4^{th} boost immunisation |
| 125 | Tail bleed |

3.2 Immunogen+Adjuvant Formulation.

Preparation of Emulsion Adjuvant AS03:

Tween 80 is dissolved in phosphate buffered saline (PBS) to give a 2% solution in the PBS. To provide 100 ml two-fold concentrate emulsion 5 g of DL alpha tocopherol and 5 ml of squalene are vortexed to mix thoroughly. 90 ml of PBS/Tween solution is added and mixed thoroughly. The resulting emulsion is then passed through a syringe and finally microfluidised by using an M110S microfluidics machine. The resulting oil droplets have a size of approximately 180 nm.

Mix adjuvant 1:1 with protein solution, vortex briefly (10 seconds at middle speed) and incubate for 10 minutes at room temperature on an orbital shaker. Vortex briefly before injection and administer 100 ul total suspension per mouse by the intramuscular route at 2 separate sites (ie. 2×50 ul per mouse, one injection in each quadriceps muscle). Prepare fresh before each immunisation.

Alum

Supplied by SIGMA (Cat. No. A-1577). Prepare a 2 mg/ml suspension of alum in PBS. Mix adjuvant 1:1 with protein solution, vortex briefly and incubate shaking gently for 10 minutes at room temperature. Vortex briefly before injection and administer 100 ul total suspension per mouse i/p. Prepare fresh before each immunisation.

CpG—ImmunEasy

Supplied by Qiagen (Cat.No. 303101). Mix the stock pot of adjuvant by gentle vortexing, then mix adjuvant 1:1 with protein by gently pipetting up and down 5 times. Incubate at room temperature for 15 minutes. Gently pipette the mix up and down 5 times and administer 100 ul suspension per mouse by the intramuscular route at 2 separate sites (ie. 2×50 ul per mouse, one injection in each quadriceps muscle). Prepare fresh before each immunisation.

CFA/IFA

Supplied by SIGMA (Cat. Nos. F-5881, F-5506). Formulate 1:1 with pre-mixed CFA for primary or IFA for boosts. Whirlimix sample to ensure an even white suspension with the CFA/IFA. Store on ice for at least 30 mins prior to use and whirlimix thoroughly prior to dosing.

3.3 Anti-mouse IL-13 Antibody Responses.

Anti-mouse IL-13 antibody responses were monitored in the serum samples using an anti-mouse IL-13 antibody detection ELISA.

96-well Maxisorp plates were coated with anti-mouse IL-13 monoclonal antibody (Cat. No. MAB, R+D Systems) in carbonate-bicarbonate buffer overnight at 4° C. Plates were then blocked with 3% BSA/TBST for 1 hour at RT, washed 3 times in TBST, and incubated with mouse IL-13 (Cat. No. 413-ML-025, R+D Systems) for 1 hour at RT. After washing, plates were incubated with mouse sera for 1 hour at RT, washed again and incubated with HRP conjugated anti-mouse IgG polyclonal antibody (SIGMA, Cat. No. A-9309). Following further washing the plates were developed with 0-phenylenediamine dihydrochloride peroxidase substrate for 30 minutes.

The level of anti-mouse IL-13 antibodies in the serum was expressed as an endpoint titre. The endpoint titre is defined as that dilution of serum that is equivalent to twice the ELISA background reading.

Anti-mouse IL-13 antibody endpoint titre

| Mouse | AS03 | Alum | CpG | CFA/IFA |
|---|---|---|---|---|
| 1 | 1/875 | 1/7250 | 1/67500 | 1/6750 |
| 2 | 1/9250 | 1/800 | 1/80000 | 1/975 |
| 3 | 1/160 | 1/9000 | 1/54000 | 1/6000 |
| 4 | 1/9000 | 1/6500 | 1/62500 | 1/16000 |
| 5 | 1/3600 | 1/10000 | 1/77500 | 1/31000 |

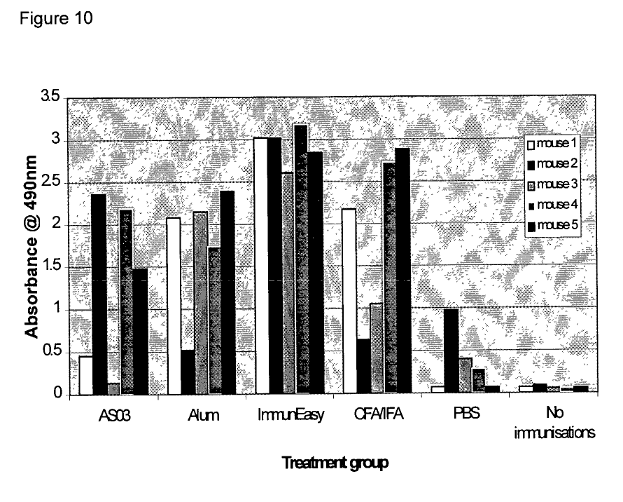

FIG. 10 illustrates the anti-mouse IL-13 antibody profiles in the various treatment groups at day 125, for serum samples diluted at 1/100.

All five mice immunised with GST-cIL-13 in combination with CpG adjuvant raised strong anti-mouse IL-13 auto-antibody responses. This is in contrast to the other adjuvants, where responses were less consistent throughout each group, some mice raising very weak responses indeed.

These results indicate that CpG adjuvant is much more effective at raising consistent high titre anti-mouse IL-13 auto-antibody responses compared to the other adjuvants tested.

These serum samples were analysed for IL-13 neutralising ability in an in vitro IL-13 neutralisation bioassay.

3.4 IL-13 Neutralisation Capacity.

To measure the ability of mouse serum to neutralise the bioactivity of recombinant mouse IL-13 on human TF-1 cells (ATCC Cat. No. CRL-2003), 5 ng/ml recombinant mouse IL-13 was incubated with various concentrations of sera for 1 hour at 37° C. in a 96-well tissue culture plate (Gibco BRL). Following this pre-incubation period, TF-1 cells were added. The assay mixture, containing various serum dilutions, recombinant mouse IL-13 and TF-1 cells, was incubated at 37° C. for 70 hours in a humidified CO₂ incubator. MTT substrate (Cat. No. G4000, Promega) was added during the final 4 hours of incubation, after which the reaction was stopped with an acid solution to solubilise the metabolised blue formazan product. The absorptance of the solution in each well was read in a 96-well plate reader at 570 nm wavelength.

Note that this assay is only able to measure mouse IL-13 neutralisation capacity in serum dilutions greater than or equivalent to 1/100. Serum dilutions less than 1/100 induce non-specific proliferative effects in TF-1 cells.

The capacity of the serum to neutralise mouse IL-13 bioactivity was expressed as, that dilution of serum required to neutralise the bioactivity of 5 ng /ml mouse IL-13 by 50% (=ND₅₀). The more dilute serum sample required, the more potent the neutralisation capacity.

The highest concentration of mouse D5 serum tested was a 1/100 dilution. This did not neutralise the bioactivity of 5 ng/ml mouse IL-13 by 50%, therefore the ND₅₀ is expressed as <1/100 dilution.

| Mouse | Mouse IL-13 |
|---|---|
| (Serum samples taken at | neutralisation capacity |
| day 125) | (ND₅₀) |
| C1 | 1/1250 |
| C2 | 1/5230 |
| C3 | 1/523 |
| C4 | 1/417 |
| C5 | 1/1670 |
| D5 | <1/100 |

Day 125 serum samples from all five mice immunised with GST-cIL-13 in combination with CpG adjuvant, were able to potently neutralise the bioactivity of mouse IL-13 in an in vitro bioassay. In contrast, the day 125 serum sample from mouse D5 (immunised with GST-cIL-13 in CFA/IFA) was unable to neutralise the bioactivity of mouse IL-13 at all dilutions tested.

These results indicate that CpG adjuvant is much more effective at raising neutralising anti-mouse IL-13 auto-antibody responses compared to the other adjuvants tested.

1. An isolated protein which is at least 30% but less than 100% identical to a human protein which polypeptide
(a) contains at least one mutation which is characteristic of an analogous non-human protein;
(b) is capable of raising antibodies in human
(c) is sufficiently structurally similar to the human protein that the antibodies bind to both the human protein and the polypeptide and;
wherein the protein is not an antibody.

2. A protein having B-cell epitopes from a mammalian self-antigen and a mutation that gives rise to a sequence of an analogous protein of a second mammalian species such that the protein is able to raise in the species from which the B-cell epitopes derived, an immune response that recognises the natural protein from which the B-cell epitopes are derived.

3. A protein having B-cell epitopes of a self-protein which are grafted, by substitution, into a frame work of an analogous protein from a second mammalian species such that the protein is able to raise in the species in which the B-cell epitopes are derived, an immune response that recognises the natural protein from which the B-cell epitopes are derived.

4. A protein as claimed in any of claims 1 to 3 comprising conserved surface region introduced into the non-surface exposed region, said mutation giving rise to a sequence of an analogous protein such that the protein is able to raise an immune response to the self protein in the species from which the self-protein is derived.

5. A protein as claimed in any one of claims 1 to 4 wherein the immune response is a neutralising antibody response.

6. A protein as claimed in any of claims 1 to 5 wherein the human protein, or B-cell epitope is derived from a cytokine.

7. A cytokine as claimed in claim 6, which is a 4-helical cytokine.

8. A cytokine as claimed in claim 7 which is IL-4 or IL-13.

9. A mutated human—IL-13 having one or more of the following substitutions or a substitution involving a conservative substitution thereof:

| | | | | | |
|---|---|---|---|---|---|
| R | → | K | at | position | 30 |
| V | → | S | at | position | 37 |
| Y | → | F | at | position | 63 |
| A | → | V | at | position | 65 |
| E | → | D | at | position | 68 |
| E | → | Y | at | position | 80 |
| K | → | R | at | position | 81 |
| M | → | I | at | position | 85 |
| G | → | H | at | position | 87 |
| Q | → | H | at | position | 113 |
| V | → | I | at | position | 115 |
| D | → | K | at | position | 117 |

10. A mutated human IL-13 as claimed in claim 9 having a plurality of substitutions as set forth in claim 9.

11. A mutated human IL-13 as claimed in any of claim 9 or 10 having one or more of the following sequences
L K E L I E E L S N
F C V A L D S L
A I Y R T Q R I L H G
K I E V A H F J T K L L
or a variant of said sequence comprising one or more conservative substitutions.

12. A mutated human IL-13 as shown in FIG. 9.

13. A polynucleotide encoding a protein of claim 1 to 12.

14. A polynucleotide of claim 13 which is a DNA and is operably linked to a promoter.

15. A vector comprising a polynucleotide of claim 13 or 14.

16. A host transformed with a polynucleotide of claim 13 or 14 or vector of claim 15.

17. A pharmaceutical composition comprising, a protein, polynucleotide, vector as claimed in any of claims 1 to 15 with a pharmaceutically acceptable carrier or excipient.

18. A pharmaceutical composition as claimed in claim 17 additionally comprising an adjuvant.

19. A pharmaceutical composition as claimed in claim 18 comprising a protein as set forth in any of claims 1 to 12 and an immunostimulatory oligonucleotide.

20. A pharmaceutical composition as claimed in claim 19 wherein the immunostimulatory oligonucleotide is selected from the group:

| | | | |
|---|---|---|---|
| OLIGO 1: | TCC ATG ACG TTC CTG ACG TT | (CpG 1826) | (SEQ ID NO:1) |
| OLIGO 2: | TCT CCC AGC GTG CGC CAT | (CpG 1758) | (SEQ ID NO:2) |
| OLIGO 3: | ACC GAT GAC GTC GCC GGT GAC GGC ACC ACG | | (SEQ ID NO:3) |
| OLIGO 4: | TCG TCG TTT TGT CGT TTT GTC GTT | (CpG 2006) | (SEQ ID NO:4) |
| OLIGO 5: | TCC ATG ACG TTC CTG ATG CT | (CpG 1668) | (SEQ ID NO:5) |

21. A protein, polynucleotide, vector, host or composition as claimed in any of claims 1 to 20 for use in medicine.

22. Use of a protein as claimed in any of claim 1 to 12 in the manufacture of a medicament for the treatment of IL-13 mediated diseases.

23. Use as claimed in claim 22 for the treatment of asthma.

24. A method for the treatment of prophylaxis of IL-13 mediated disease comprising the administration of a safe and effective amount of composition according to any of claims 17-20 to a patient in need thereof.

25. A method for the preparation of a protein according to any of claims 1 to 12 which method comprises:
1. identification of one or more regions of a self, typically human, protein against which an antibody response is desired.
2. identification of the amino-acid sequence of the self protein.
3. identification of the amino-acid sequence of an analogous protein construction by recombinant DNA techniques of a chimaeric molecule containing at least one target region identified in step 1, whose amino-acid sequence is taken from the sequence identified in step 2, and
sufficient amino-acids from the sequence(s) identified in step 3 to enable the resulting protein to fold into a shape similar to that the self protein such that the mutated protein can raise an immune response that recognises the self protein.


SEQUENCE LISTING (25 sequences)

SEQ ID NO: 1 - protein, 10 aa, Artificial Sequence
  mutated epitope
LKELIEELSN

SEQ ID NO: 2 - protein, 8 aa, Artificial Sequence
  mutated epitope
FCVALDSL

SEQ ID NO: 3 - protein, 11 aa, Artificial Sequence
  mutated epitope
AIYRTQRILHG

SEQ ID NO: 4 - protein, 12 aa, Artificial Sequence
  mutated epitope
KIEVAHFITKLL

SEQ ID NO: 5 - DNA, 20 bp, unknown
  synthetic immunostimulatory oligonucleotide
tccatgacgttcctgacgtt

SEQ ID NO: 6 - DNA, 18 bp, unknown
  synthetic immunostimulatory oligonucleotide
tctcccagcgtgcgccat

SEQ ID NO: 7 - DNA, 30 bp, unknown
  synthetic immunostimulatory oligonucleotide
accgatgacgtcgccggtgacggcaccacg

SEQ ID NO: 8 - DNA, 24 bp, unknown
  synthetic immunostimulatory oligonucleotide
tcgtcgttttgtcgttttgtcgtt

SEQ ID NO: 9 - DNA, 20 bp, unknown
  synthetic immunostimulatory oligonucleotide
tccatgacgttcctgatgct

SEQ ID NO: 10 - DNA, 72 bp, Artificial Sequence
  PCR oligoprimer for Chimeric IL13 for murine use
tgtgatgttgaccagctcctcaatgagctccctaagggtcagagggagagacacagatcttggcaccggccc

SEQ ID NO: 11 - DNA, 73 bp, Artificial Sequence
  PCR oligoprimer for Chimeric IL13 for murine use
aggagctggtcaacatcacacaagaccagactcccctgtgcaacggcagcatggtatggagtgtggacctggc

SEQ ID NO: 12 - DNA, 72 bp, Artificial Sequence
  PCR oligoprimer for Chimeric IL13 for murine use
gcaattggagatgttggtcagggattccagggctgcacagtacccgccagcggccaggtccacactccatac

SEQ ID NO: 13 - DNA, 73 bp, Artificial Sequence
  PCR oligoprimer for Chimeric IL13 for murine use
tgaccaacatctccaattgcaatgccatcgagaagacccagaggatgctgggcggactctgtaaccgcaaggc

SEQ ID NO: 14 - DNA, 72 bp, Artificial Sequence
  PCR oligoprimer for Chimeric IL13 for murine use
aaactgggccacctcgattttggtatcggggaggctggagaccgtagtgggggccttgcggttacagagtcc

SEQ ID NO: 15 - DNA, 71 bp, Artificial Sequence
  PCR oligoprimer for Chimeric IL13 for murine use
aaatcgaggtggcccagtttgtaaaggacctgctcagctacacaaagcaactgtttcgccacggccccttc

SEQ ID NO: 16 - DNA, 28 bp, Artificial Sequence
  PCR oligoprimer for Chimeric IL13 for murine use
cgcggattcgggccggtgccaagatctg

SEQ ID NO: 17 - DNA, 37 bp, Artificial Sequence
  PCR oligoprimer for Chimeric IL13 for murine use
ctccgctcgagtcgacttagaaggggccgtggcgaaa

SEQ ID NO: 18 - DNA, 28 bp, Artificial Sequence
  PCR oligoprimer for Chimeric IL13 for murine use
cgcggatccgggccggtgccaagatctg

SEQ ID NO: 19 - DNA, 336 bp, Artificial Sequence
  Chimeric IL13 for murine use
gggccggtgccaagatctgtgtctctccctctgacccttagggagctcattgaggagctggtcaacatcacacaagaccagactcccctgtgcaacggcagcatggtatggagtgtggacctggccgctggcgggtactgtgcagccctggaatccctgaccaacatctccaattgcaatgccatcgagaagacccagaggatgctgggcggactctgtaaccgcaaggcccccactacggtctccagcctccccgataccaaaatcgaggtggcccagtttgtaaaggacctgctcagctacacaaagcaactgtttcgccacggccccttctaa

SEQ ID NO: 20 - protein, 111 aa, Artificial Sequence
  Chimeric IL13 for murine use
GPVPRSVSLPLTLRELIEELVNITQDQTPLCNGSMVWSVDLAAGGYCAALESLTNISNCNAIEKTQRMLGGLCNRKAPTTVSSLPDTKIEVAQFVKDLLSYTKQLFRHGPF

SEQ ID NO: 21 - DNA, 399 bp, Artificial Sequence
  Chimeric IL13 for human use
atggcgcttttgttgaccacggtcattgctctcacttgccttggcggctttgcctccccaggccctgtgcctccctctacagcccttaaggagcttattgaggagctgagcaacatcacccagaaccagaaggctccgctctgcaatggcagcatggtttggagcatcaacctgacagctggcatgttctgtgtagccctggattccctgatcaacgtgtcaggctgcagtgccatctacaggacccagaggatattgcatggcttctgcccgcacaaggtctcagctgggcagttttccagcttgcatgtccgagacaccaaaatcgaagtagcccactttataacaaaactgctcttacatttaaagaaactttttcgcgagggacggttcaactga

SEQ ID NO: 22 - protein, 132 aa, Artificial Sequence
  Chimeric IL13 for human use
MALLLTTVIALTCLGGFASPGPVPPSTALKELIEELSNITQNQKAPLCNGSMVWSINLTAGMFCVALDSLINVSGCSAIYRTQRILHGFCPHKVSAGQFSSLHVRDTKIEVAHFITKLLLHLKKLFREGRFN

SEQ ID NO: 23 - DNA, 396 bp, Artificial Sequence
  Chimeric IL13 for murine use
atggcgctctgggtgactgcagtcctggctcttgcttgccttggtggtctcgccgccccagggccggtgccaagatctgtgtctctccctgtgacccttaaggagcttattgaggagctgaccaacatcacacaagaccagactcccctgtgcaacggcagcatggtatggagtgtggacctggccgctggcgggttctgtgtagccctggattccctgaccaacatctccaattgcaatgccatcttcaggacccagaggatattgcatgccctctgtaaccgcaaggcccccactacggtctccagcctccccgataccaaaatcgaagtagcccactttataacaaaactgctcacctacacaaagaacctgtttcgccgcggccccttctaa

SEQ ID NO: 24 - protein, 131 aa, Artificial Sequence
  Chimeric IL13 for murine use
MALWVTAVLALACLGGLAAPGPVPRSVSLPVTLKELIEELTNITQDQTPLCNGSMVWSVDLAAGGFCVALDSLTNISNCNAIFRTQRILHALCNRKAPTTVSSLPDTKIEVAHFITKLLTYTKNLFRRGPF

SEQ ID NO: 25 - protein, 150 aa, Artificial Sequence
  Chimeric IL4 for human use
MGLTSQLLPPLFFLLACAGNFVHGHKCDKNHLREIIGILNEVTGEKTLCTELTVTDIFAASKNTTESELVCRASKVLRIFYLKHEKDTRCLGATAKNSSVLMELQRLFRAFRCLDGLNSCPVKEANQSSLKDFLESLKSIMQMDYSKCSS